SEQUENCE LISTING (16 sequences)

SEQ ID NO: 1 - protein, 9 aa, Bos sp.
DIQYLPLIK

SEQ ID NO: 2 - protein, 22 aa, Bos sp.
AIQISYNPDQPSKPNNIESATK

SEQ ID NO: 3 - protein, 25 aa, synthetic construct
  VARIANT 25: note = Residue may be deleted
DVLMTQTPLSLPVSLGDQASISCRS

SEQ ID NO: 4 - protein, 36 aa, synthetic construct
  VARIANT 1: note = Residue may be deleted
  VARIANT 36: note = Residue may be deleted
RADAAPTVSIFPPSSEQLTSGGASVVCFLNNFYPKD

SEQ ID NO: 5 - protein, 16 aa, synthetic construct
  VARIANT 1: note = Residue may be deleted
  VARIANT 16: note = Residue may be deleted
RQNGVLNSWTDQDSKD

SEQ ID NO: 6 - protein, 29 aa, synthetic construct
  VARIANT 1: note = Residue may be deleted
  VARIANT 29: note = Residue may be deleted
RVEAEDLGVYYCFQGSHVOKTFGAGGTKL

SEQ ID NO: 7 - protein, 10 aa, synthetic construct
  VARIANT 1: note = Residue may be deleted
  VARIANT 10: note = Residue may be deleted
KTSTSPIVKS

SEQ ID NO: 8 - protein, 18 aa, synthetic construct
  VARIANT 1: note = Residue may be deleted
  VARIANT 18: note = Residue may be deleted
KAPQVYTIPPPKEQMAKD

SEQ ID NO: 9 - protein, 14 aa, synthetic construct
  VARIANT 1: note = Residue may be deleted
  VARIANT 14: note = Residue may be deleted
RVNSAAFPAPIEKT

SEQ ID NO: 10 - protein, 12 aa, synthetic construct
  VARIANT 1: note = Residue may be deleted
  VARIANT 12: note = Residue may be deleted
KDVLTITLTPKV

SEQ ID NO: 11 - protein, 8 aa, synthetic construct
  VARIANT 1: note = Residue may be deleted
  VARIANT 8: note = Residue may be deleted
KSQVFLKM

SEQ ID NO: 12 - protein, 20 aa, synthetic construct
  VARIANT 1: note = Residue may be deleted
  VARIANT 20: note = Residue may be deleted
KQYFAYWGQGTLVTVSAAKT

SEQ ID NO: 13 - protein, 5 aa, synthetic construct
QVQLK

SEQ ID NO: 14 - protein, 10 aa, synthetic construct
DVLTITLTPK

SEQ ID NO: 15 - protein, 34 aa, synthetic construct
VTCVVVDISKDDPEVQFSWFVDDVEVHTAHTQPR

SEQ ID NO: 16 - protein, 9 aa, synthetic construct
EEQFNSTFR

(19) United States
(12) Patent Application Publication
(10) Pub. No.: US 2024/0159628 A1
(43) Pub. Date: May 16, 2024

(54) PRESSED AND MIXED THIN FILM ENZYMATIC REACTOR AND METHODS OF MAKING THE SAME

(71) Applicant: Waters Technologies Corporation, Milford, MA (US)

(72) Inventor: Lindsay Morrison, Waltham, MA (US)

(21) Appl. No.: 18/508,933

(22) Filed: Nov. 14, 2023

Related U.S. Application Data
(60) Provisional application No. 63/383,779, filed on Nov. 15, 2022.

(51) Int. Cl. G01N 1/28

(52) U.S. Cl. CPC G01N 1/2813

(57) ABSTRACT

Disclosed herein are methods and devices for accelerating substrate-enzyme reaction rates for sample preparation in bioprocessing assays using pressed and mixed thin films containing predetermined ratios of a biological substrate, e.g., polypeptide, protein, nucleic acid, sugar, or lipid, and an enzyme.

### CROSS-REFERENCE TO RELATED APPLICATIONS

This patent application is a U.S. Nonprovisional Application which claims priority to and the benefit of U.S. Provisional Application No. 63/383,779, filed Nov. 15, 2022, the contents of which are hereby incorporated herein by reference in their entirety.

### SEQUENCE LISTING

This patent application contains a Sequence Listing which has been submitted electronically in XML format and is hereby incorporated by reference in its entirety. The XML, copy, created Nov. 9, 2023, is named WAC-399US_SL.xml and is 22,038 bytes in size.

### BACKGROUND

The time required for preparative reactions for analytical techniques, particularly in the bioprocessing space, can add significant delays to workflows. These delays are prohibitive to obtaining real-time results, an important requirement in feedback loop-modulated bioreactor design. Acceleration of reactions in microdroplets has been demonstrated for small molecules. Four postulated mechanisms exist for the acceleration of small molecule reactions in microdroplets: (1) dipole alignment and partial solvation at the droplet surface; (2) concentration effects due to localization and confinement at the droplet surface; (3) concentration effects due to droplet evaporation; and (4) effects of charge and electric fields. However, due to the preferential localization of folded proteins in the interior of droplets, only spatial confinement, evaporation effects, and electric field impacts are observed for protein-enzyme reactions. Consequently, the observed acceleration factors are in the 10³-10⁴ range, rather than the 10⁵-10⁶ range reported for some small molecule reactions. One limiting parameter of microdroplets is the inherently short lifetime of such species. Thin films have also been used for reaction acceleration and are effective for similar reasons as microdroplets. Although the acceleration factors observed for films has generally been lower than for microdroplets, the lifetime of films can be extended many orders of magnitude longer than microdroplets, affording a simple way to overcome the reduction in acceleration factor. The present disclosure provides pressed and mixed thin films and methods of making the same as a solution to accelerating reaction rates of for substrate-enzyme systems (SES).

### SUMMARY OF THE DISCLOSURE

Disclosed herein, in certain embodiments, are methods for accelerating reaction rates of substrate-enzyme systems (SES), comprising preparing pressed and mixed thin films comprising predetermined ratios of a substrate (e.g., a polypeptide, protein, nucleic acid, sugar, or lipid) and enzyme. Also disclosed are devices for producing said thin films.

In an aspect, disclosed herein is a method of preparing a protein sample in a thin film for a bioprocessing assay, comprising: (a) combining an enzyme and a protein substrate at a predetermined ratio to produce an substrate-enzyme mixture (e.g., SES); (b) depositing the mixture of step (a) in a bottom conical or frustoconical surface; and (c) pressing the mixture between the bottom conical or frustoconical surface and a top conical or frustoconical surface by applying a predetermined force between the top conical or frustoconical surface and the bottom conical or frustoconical surface, wherein the top conical or frustoconical surface and bottom conical or frustoconical surface are vertically oriented and configured to produce a nested conical or frustoconical interface; thereby forming a thin film comprising the substrate-enzyme (e.g., protein-enzyme) mixture in the nested conical interface. In certain embodiments, the predetermined ratio of the enzyme and substrate (e.g., protein) is about 1:20, 1:25, 1:30, 1:40, 1:50, 1:60, 1:70, 1:80, 1:90, 1:100, 1:110, 1:120, 1:130, 1:140, 1:150, 1:160, 1:170, 1:180, 1:190, or 1:200 (mol enzyme:mol substrate). In certain embodiments, the substrate-enzyme mixture is in a solution at a concentration of 0.1 to 20 mg/mL (e.g., 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, and 20 mg/mL). In certain embodiments, the substrate-enzyme mixture is in 10 to 100 μL (e.g., 10 to 100 μL, 15 to 95 μL, 20 to 90 μL, 25 to 85 μL, 30 to 80 μL, 35 to 75 μL, 40 to 70 μL, 45 to 65 μL, and 50 to 60 μL) of the solution. In certain embodiments, the solution containing the substrate-enzyme system has a viscosity of 0.1 to 2.0 mPa-S (e.g., 0.1 to 2.0 mPa-S, 0.2 to 1.9 mPa-S, 0.3 to 1.8 mPa-S, 0.4 to 1.7 mPa-S, 0.5 to 1.6 mPa-S, 0.6 to 1.5 mPa-S, 0.7 to 1.4 mPa-S, 0.8 to 1.3 mPa-S, 0.9 to 1.2 mPa-S, and 1.0 to 1.1 mPa-S). In certain embodiments, the predetermined force is between 3 and 20 pounds (e.g., 3 to 20 lbs, 4 to 19 lbs, 5 to 18 lbs, 6 to 17 lbs, 7 to 16 lbs, 8 to 15 lbs, 9 to 14 lbs, 10 to 13 lbs, and 11 to 12 lbs). In certain embodiments, the predetermined force is between 10 and 15 pounds (e.g., 10 to 15 lbs, 11 to 14 lbs, and 12 to 13 lbs). In certain embodiments, the predetermined force is applied for a duration between 3 and 5 s (e.g., 3.0 to 5.0 s, 3.1 to 4.9 s, 3.2 to 4.8 s, 3.3 to 4.7 s, 3.4 to 4.6 s, 3.5 to 4.5 s, 3.6 to 4.4 s, 3.7 to 4.3 s, 3.8 to 4.2 s, and 3.9 to 4.1 s). In certain embodiments, the application of force is followed by withdrawal of force for a duration of between 1 s and 2 s (e.g., 1.0 to 2.0 s, 1.1 to 1.9 s. 1.2 to 1.8 s, 1.3 to 1.7 s, and 1.4 to 1.6 s). In certain embodiments, the application and withdrawal of force is performed over a total duration of about 30 s. In certain embodiments, the method increases a reaction rate between the enzyme and the substrate (e.g., protein) as compared to a reaction rate between the enzyme and the substrate in bulk solution. In certain embodiments, the increase in the reaction rate results from an increase in the rate of binding of the enzyme to the substrate (Kₒₙ). In certain embodiments, the enzyme is selected from the group consisting of a protease, a glycosylase, and a phosphatase. In certain embodiments, the protease is trypsin, chymotrypsin, immunoglobulin degrading enzyme (IgDE), immunoglobulin G-degrading enzyme of Streptococcus pyogenes (IdeS), Asp-N protease, or Lys-C protease. In certain embodiments, the glycosylase is peptide:N-glycosidase F (PNGase F). In certain embodiments, the phosphatase is alkaline phosphatase (ALP). In certain embodiments, the bioprocessing assay is liquid chromatography (LC), mass spectrometry (MS), or a combination thereof. In certain embodiments, the bioprocessing assay is peptide mapping, peptide monitoring, proteomics, protein quantification, glycan characterization, glycomics, and glycoprotein characterization. In certain embodiments, the thin film has a thickness no greater than about 100 μm, about 90 μm, about 80 μm, about 70 μm, about 60 μm, about 50 μm, about 40 μm, about 30 μm, about 20 μm, or about 10 μm. In certain embodiments, the thin film has a thickness no greater than about 50 μm.

In an aspect, disclosed herein is an apparatus for production of a thin film (e.g., a thin film comprising a SES), comprising (a) a vertically-oriented, convex, top conical or frustoconical surface, wherein the base of the top conical or frustoconical surface is attached to a top base member; (b) a vertically-oriented, concave, bottom conical or frustoconical surface configured to hold a sample volume, wherein the bottom conical or frustoconical surface is inside of a cylindrical bottom base member; (c) a stationary linear actuator configured to move along a vertical axis and operably connected to a raised cylindrical surface on the top base member or operably connected to the bottom base member; (d) a fluid inlet port operably connected to the top conical or frustoconical surface and/or the bottom conical or frustoconical surface; (e) one or more (e.g., 1, 2, 3, 4, or more) one or more heating elements (e.g., cartridge heaters or an infrared laser) operably connected to a temperature controller and relay circuit; (f) a thermistor configured for placement inside a thermistor hole in the raised cylindrical surface of the top base member, said thermistor being operably connected to the temperature controller; wherein the top conical or frustoconical surface and the bottom conical or frustoconical surface are configured to produce a nested conical or frustoconical interface. In certain embodiments, the thin film is configured for deposition of one or more (e.g., 1, 2, or more) biological agents (e.g., enzyme and enzyme substrate, such as a protein, nucleic acid, sugar, or lipid) on a surface of the thin film. In certain embodiments, the one or more biological agents is a protein of interest. In certain embodiments, the protein of interest is an enzyme. In certain embodiments, the one or more biological agents further includes a substrate of the enzyme. In certain embodiments, the linear actuator is configured to generate a predetermined force between the top conical or frustoconical surface and the bottom conical or frustoconical surface that is between 3 and 20 pounds e.g., 3 to 20 lbs, 4 to 19 lbs, 5 to 18 lbs, 6 to 17 lbs, 7 to 16 lbs, 8 to 15 lbs, 9 to 14 lbs, 10 to 13 lbs, and 11 to 12 lbs). In certain embodiments, the linear actuator is configured to generate angular movement at an offset angle of 10°-30°. In certain embodiments, the top and/or bottom conical or frustoconical surface each has a slant height of 3 to 15 cm (e.g., 3, 4, 5, 6, 7, 8, 9, 10, 11, 12, 13, 14, and 15 cm). In certain embodiments, the bottom conical or frustoconical surface has a vertical height of 2.85 cm to 10 cm (e.g., 2.85, 3, 4, 5, 6, 7, 8, 9, and 10 cm). In certain embodiments, the top conical or frustoconical surface and the bottom conical or frustoconical surface each have a vertical angle (e.g., slant angle) of 45°. In certain embodiments, the top frustoconical surface and the bottom frustoconical surface comprise an upper base having a diameter of 1 mm to 10 mm (e.g., 1, 2, 3, 4, 5, 6, 7, 8, 9, and 10 cm). In certain embodiments, each of the top conical or frustoconical surface and the bottom conical or frustoconical surface is independently composed of a material selected from the group consisting of stainless steel, poly(methyl acrylate), and glass. In certain embodiments, the fluid inlet port is operably connected to a sample injection device. In certain embodiments, the top conical or frustoconical surface is actuated and the bottom conical or frustoconical surface is stationary. In certain embodiments, the bottom conical or frustoconical surface is actuated and the top conical or frustoconical surface is stationary. In certain embodiments, the bottom conical or frustoconical surface is configured to hold a fluid volume of at least between 10 μL and 100 μL (e.g., 10 to 100 μL, 15 to 95 μL, 20 to 90 μL, 25 to 85 μL, 30 to 80 μL, 35 to 75 μL, 40 to 70 μL, 45 to 65 μL, and 50 to 60 μL). In certain embodiments, the thin film has a thickness no greater than about 100 μm, about 90 μm, about 80 μm, about 70 μm, about 60 μm, about 50 μm, about 40 μm, about 30 μm, about 20 μm, or about 10 μm. In certain embodiments, the thin film has a thickness no greater than about 50 μm.

### DEFINITIONS

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “a protein” includes a single protein or a plurality of proteins.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000-fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the phrase “biological agent” refers to any compound or molecule that exerts a biological effect. Non-limiting examples of biological agents include a protein (e.g., enzyme), a substrate for the protein (e.g., a molecule or a compound, such as sugar, lipid, fat, polypeptide or protein, nucleic acid, or others), and other biological molecules.

As used herein, the phrase “bioprocessing assay” refers to any assay that receives a biological sample as an input and performs one of multiple potential operations on the sample, including, but not limited to, dilution, concentration, isolation, mixing, digestion, constitution, reconstitution, aggregation, identification, ontology, sequencing, formulation, among others. Non-limiting examples of a bioprocessing assay include peptide mapping, peptide monitoring, proteomics, protein quantification, glycan characterization, glycomics, and glycoprotein characterization.

As used herein, the phrase “nested conical or frustoconical interface” refers to the boundary between a top conical or frustoconical surface and a bottom conical or frustoconical surface of the disclosure. The nested conical or frustoconical interface is formed in cases where the top and bottom conical or frustoconical surfaces are in sufficiently close proximity (e.g., no more than 1-30 μm) to one another so as to be capable of producing a thin film of the disclosure.

As used herein, the phrase “operably connected,” when referring to elements of an apparatus of the disclosure, means that the particular elements communicate, are attached, or are otherwise connected, physically or functionally, in such a way that they cooperate to achieve their intended function(s). “Operably connected” elements may be connected directly, indirectly, physically, or remotely.

As used herein, the term “reaction rate” refers to the speed of at which an enzymatic reaction between an enzyme and its substrate molecule (e.g., polypeptide, protein, nucleic acid, sugar, or lipid) takes place. The reaction rate can be measured, in certain embodiments, by the rate at which a concentration of a reaction product accumulates or by the rate at which the substrate (i.e., reactant) is depleted per unit time. A reaction rate can refer to the rate of a single reaction between a substrate and enzyme, the combined rate of individual sub-reactions within a reaction chain, or the rate at which a given sub-reaction within a reaction chain occurs. For example, the total reaction rate of a substrate-enzyme reaction can be divided into: (1) the rate of binding between the substrate and enzyme, termed km, and the catalytic rate constant of the enzyme per se, termed k_{cat}. At high substrate concentrations, k_{cat} tends to be rate-limiting, whereas at lower substrate concentrations diffusional processes dramatically reduce kₒₙ.

As used herein, the term “sample injection device” refers to any device configured to deliver (e.g., inject) a sample, such as a liquid sample containing an enzyme and a substrate, at a specified volume and rate and for a specified duration to an apparatus of the disclosure, or a component thereof. Generally, a sample injection device may include a means for holding the sample, such a sample collection structure, a means to measure and regulate the temperature of the sample (e.g., thermometer, thermoregulator, and heater elements), a means to pump said sample to the apparatus (e.g., a motor pump and tubing), and optionally a means to retrieve said sample from the apparatus.

As used herein, the term “substrate” refers to a biological molecule, complex, or aggregate (e.g., polypeptide, protein, nucleic acid, sugar, or lipid) which acts as a reactant in a chemical reaction involving an enzyme or enzyme complex. For example, in chemical reaction comprising a protease (e.g., trypsin) and a target molecule to be digested (e.g., arbitrary polypeptide or protein), trypsin is the enzyme and the target molecule to be digested is the substrate.

As used herein, the terms “substrate-enzyme system” and “SES” refer to any mixture comprising an enzyme and its natural substrate (e.g., peptide, protein, nucleic acid, sugar, or lipid). Non-limiting examples of an SES include a bulk aqueous solution or a thin film comprising an enzyme and its substrate in any amount.

As used herein, the term “upper base,” when referring to a frustoconical surface refers to the base of a frustoconical shape (e.g., a frustum) having the smaller diameter. A frustum is essentially a cone or pyramid in which a portion of the cone or solid is cut by a plane parallel to the base of the cone or pyramid, effectively creating a second, smaller base in the cone or pyramid. The “lower base” of a frustoconical shape is the base having the larger diameter (i.e., the base of the cone or pyramid from which the frustoconical shape is created),

### BRIEF DESCRIPTION OF THE DRAWINGS

FIGS. 1A-1D show schematic diagrams of a top base member of an apparatus for producing pressed and mixed thin films of the disclosure, the top base member having a convex, frustoconical surface. (FIG. 1A) Top view of a top base member having a central raised cylindrical surface which can be operably connected to a stationary linear actuator. The raised cylindrical surface can be fitted to attach to the linear actuator. The lateral raised surfaces have holes which can be fitted with one or more heating elements for heating the assembly. (FIG. 1B) Lateral cross-section of the top base member showing a frustoconical surface. Diagonal lines correspond to a solid interior of the top base member. Central hole can be used as an attachment site for a stationary linear actuator. (FIG. 1C) 3-dimensional (3D) view of a top base member having a frustoconical surface. (FIG. 1D) 3D view of a cross-section of a top base member having a frustoconical surface. All measurements are shown in inches.

FIGS. 2A-2B show schematic diagrams of a top base member of an apparatus for producing pressed and mixed thin films of the disclosure, the top base member having a convex, conical surface. (FIG. 2A) Top view of a top base member having a central raised cylindrical surface which can be operably connected to a stationary linear actuator. The raised cylindrical surface can be fitted to attach to the actuator. The four holes surrounding the raised cylindrical surface can be fitted to house one or more heating elements for heating the assembly. (FIG. 2B) Lateral view of the conical top base member. All measurements are shown in inches.

FIGS. 3A-3D show schematic diagrams of a bottom base member of an apparatus for producing pressed and mixed thin films of the disclosure, the top base member having a concave, frustoconical surface. (FIG. 3A) Top view of a bottom base member having a concave frustoconical surface. Central circle corresponds to the upper base of the frustoconical surface (i.e., bottom of the interior of the bottom base member). The two lateral holes correspond to housings fitted to accommodate one or more heating elements for heating the assembly. (FIG. 3B) Lateral view of a cross section of a bottom base member having a concave, frustoconical surface. Diagonal lines correspond to a solid interior of the bottom base member. The hole at the top of the figure (i.e., side of the bottom base member), which connects via a tapped hole into the upper base of the frustoconical surface of the bottom base member, corresponds to the fluid inlet port and is used for delivery of a biological sample onto the frustoconical surface of the bottom base member via a fluid inlet channel. (FIG. 3C) Lateral view of a bottom base member having a concave frustoconical surface. (FIG. 3D) Lateral cross-section of a bottom base member having a concave frustoconical surface, showing the fluid inlet port through which a biological sample is channeled to the frustoconical surface of the bottom base member. All measurements are shown in inches.

FIG. 4 shows a schematic diagram having an angled view of a bottom base member having a concave conical surface.

FIGS. 5A-5C show schematic 3D diagrams of an assembly comprising top and bottom base members having frustoconical surfaces. (FIG. 5A) Top base member shown hovering above the bottom base member. (FIG. 5B) Top base member shown nested inside of the bottom base member. (FIG. 5C) Cross-section of a top base member shown nested inside of the bottom base member.

FIGS. 6A-6C show schematic 3D diagrams of an assembly comprising top and bottom base members having conical surfaces. (FIG. 6A) Top base member shown hovering above the bottom base member. (FIG. 6B) Top base member shown nested inside of the bottom base member. (FIG. 6C) Cross-section of a top base member shown nested inside of the bottom base member.

FIGS. 7A-7B show illustrations and images of an apparatus for the production of mixed thin films of the disclosure. (FIG. 7A) Schematic diagram of a representative apparatus of the disclosure. In this schematic, solution comprising a mixture containing the substrate-enzyme system (SES) is injected through a fluid port in the top base member and rests on the bottom conical surface. Application of force between the top and bottom base members brings the top and bottom conical surfaces in close proximity to create a nested conical interface in which a thin film containing the SES is formed. (FIG. 7B) photograph of a representative apparatus of the disclosure. The apparatus includes top and bottom base members having conical surfaces. The top base member is attached to a linear actuator that brings the top and bottom base members in close proximity. The top member also includes cartridge heaters nested inside of heater holes in the top base members, said heaters being operably connected to a relay circuit that includes a thermistor and thermoregulator.

FIGS. 8A-8B are a series of plots showing digestion of bovine fibrinogen with trypsin using pressed thin films prepared using static mixing or bulk solution prepared from the same volume of starting material, i.e., a solution containing the SES. (FIG. 8A) Lineweaver-Burke plot for the “DIQYLPLIK” peptide of bovine fibrinogen (SEQ ID NO: 1) digested using a preparation containing 1 mg/mL or 8 mg/mL of a pressed thin film or control bulk solution containing the trypsin-fibrinogen system. (FIG. 8B) Lineweaver-Burke plot for the “AIQISYNPDQPSKPNNIESATK” peptide of bovine fibrinogen (SEQ ID NO: 2) digested using a preparation containing 1 mg/mL or 8 mg/mL of a pressed thin film or control bulk solution containing the trypsin-fibrinogen system. The x-intercept of the Lineweaver-Burke plot equals −1/Kₘ, where Kₘ is the Michaelis constant; the y-intercept corresponds to 1/Vₘₐₓ, where Vₘₐₓ is the maximum rate of an enzyme-catalyzed reaction; and the slope of the plot is proportional to Kₘ/Vₘₐₓ. These plots demonstrate the independence of V₀ on substrate concentration in the thin film and show that for certain peptides, the Vₘₐₓ of the reaction is substantially increased with the use of a thin film preparation as compared to a control bulk solution. The Vₘₐₓ calculated for DIQYLPLIK (SEQ ID NO: 1) in the thin film was 11.1 counts/s, compared to 2.2 counts/s in bulk solution. The Vₘₐₓ for AIQISYNPDQPSKPNNIESATK (SEQ ID NO: 2) was 12.6 counts/s in the thin film compared to 0.3 counts/s in bulk solution.

FIG. 9 is a bar graph showing peptide abundance for multiple digested peptide fragments of an antibody digested with trypsin using static or dynamic thin films or a control bulk solution over the course of 16 minutes. Tested peptide fragments include, from left to right: (−)DVLMTQTPLSLPVSLGDQASISCR(S) (SEQ ID NO: 3), (R)ADAAPTVSIFPPSSEQLTSGGASVVCFLNNFYPK(D) (SEQ ID NO: 4), (R)QNGVLNSWTDQDSK(D) (SEQ ID NO: 5), (R)VEAEDLGVYYCFQGSHVOKTFGAGGTK(L) (SEQ ID NO: 6), (K)TSTSPIVK(S) (SEQ ID NO: 7), (K)APQVYTIPPPKEQMAK(D) (SEQ ID NO: 8), (R)VNSAAFPAPIEK(T) (SEQ ID NO: 9), (K)DVLTITLTPK(V) (SEQ ID NO: 10), (K)SQVFLK(M) (SEQ ID NO: 11), and (K)QYFAYWGQGTLVTVSAAK(T) (SEQ ID NO: 12). Amino acid positions denoted by parentheses are not part of the peptide fragments, but correspond to preceding and succeeding amino acids from the full-length polypeptide from which they are generated (“(−)” denotes no preceding or succeeding amino acid at the position shown).

FIGS. 10A-10D are plots showing digestion kinetics of selected peptides described in FIG. 9, above, over the course of 16 minutes using static thin film preparation, dynamic thin film preparation, of a control bulk solution, as measured by the abundance of each peptide species, which include (R)QNGVLNSWTDQDSK(D) (SEQ ID NO: 5) (FIG. 10A), (K)APQVYTIPPPKEQMAK(D) (SEQ ID NO: 8) (FIG. 10B), (R)VNSAAFPAPIEK(T) (SEQ ID NO: 9) (FIG. 10C), and (R)VEAEDLGVYYCFQGSHVOKTFGAGGTK(L) (SEQ ID NO: 6) (FIG. 10D). These plots demonstrate improved digestion of the antibody using static and dynamic films, as compared to control solution, with dynamic films showing the fastest digestion kinetics.

FIG. 11 shows a deconvolved mass spectrum showing the large peptidic fragments of tryptic digestion in bulk solution (top), digestion in a thin film (middle) and digestion in a dynamic thin film (bottom). The abundance of the large 36 kDa fragment is notably reduced in the thin film and dynamic thin film digestions. These digestions instead feature numerous smaller fragments in the 5-10 kDa range. This indicates improved digestion of the intact protein into smaller intermediates in the thin film conditions.

FIG. 12 is a bar graph showing peptide abundance of a histogram showing the number of peptides of the given abundance for each digestion technique. The thin film and dynamic thin film generate more peptides in general, but especially for relatively low abundance peptides having intensities below 10,000.

FIGS. 13A-13D show plots illustrating the yield of four peptides, QVQLK (SEQ ID NO: 13; FIG. 13A), DVLTITLTPK (SEQ ID NO: 14; FIG. 13B), VTCVVVDISKDDPEVQFSWFVDDVEVHTAHTQPR (SEQ ID NO: 15; FIG. 13C), and EEQFNSTFR (SEQ ID NO: 16; FIG. 13D), from a one-pot thin-film digest of a reference monoclonal antibody (Waters Mass Check Standard) as a function of film thickness.

### DETAILED DESCRIPTION

Disclosed herein, in certain embodiments, are methods for accelerating reaction rates of substrate-enzyme systems (SES), comprising preparing pressed and mixed thin films comprising predetermined ratios of a substrate (e.g., polypeptide, protein, nucleic acid, sugar, or lipid) and an enzyme that acts on the substrate (e.g., a protease, nuclease, glycosylase, lipase, kinase, phosphatase, etc.). Such methods are useful, e.g., for sample preparation in a variety of bioanalytical or bioprocessing assays (e.g., peptide mapping, peptide monitoring, proteomics, protein quantification, glycan characterization, glycomics, and glycoprotein characterization). Also disclosed are apparatuses for producing the pressed and mixed thin films. The sections that follow describe the invention in greater detail.

### Reaction Rates in Substrate-Enzyme Systems

Substrate-enzyme systems (SES) are simple or complex mixtures of an enzyme and its corresponding substrate (e.g., polypeptide, protein, nucleic acid, sugar, or lipid), such as a solution containing the substrate and enzyme. Reaction rates in a SES are given by two separate rate constants, the rate of binding of the substrate and enzyme complex, termed kₒₙ, and the catalytic rate constant of the enzyme, termed k_{cat}. At high substrate concentrations, k_{cat} tends to be rate-limiting, whereas at lower substrate concentrations diffusional processes dramatically reduce kₒₙ.

Most common, industrial, enzymatic reactions are carried out in bulk reaction systems, ordinarily in batch reactions, in which the overall reaction rate is largely limited by diffusion of the enzymes and substrates, i.e., kₒₙ. Efforts to increase reaction rates of such reactions have employed the use of microdroplets. The acceleration of reaction rates in microdroplets can largely be attributed to improvement made to the kₒₙ rate due to spatial confinement and droplet dehydration. A limiting feature of microdroplets is their inherently short lifetime. Thin films, on the other hand, exhibit a lifetime many orders of magnitude longer than microdroplets, thereby providing a potential avenue for accelerating reaction rates in SES. Disclosed herein, in certain embodiments, are thin films and methods of making the same for accelerating reaction rates of SES, thereby improving the efficiency and productivity of sample preparation in bioprocessing and bioanalytical assays.

### Thin Films

Thin films are material substrates formulated in thin layers, typically having a thickness ranging between less than a nanometer (nm) to several micrometers (μm). These films have a broad range of applications that include, without limitation, use in magnetic recording media, semiconductor devices, integrated passive devices, light emitting diodes (LEDs), optical coatings, coatings on cutting tools, solar cells, batteries, as well as pharmaceutical drug delivery systems. Methods for the production of thin films include, but are not limited to, spray deposition, centrifugal spread, and physical pressing. Centrifugal spreading devices are common and afford simple implementation, as a volume of a liquid can be placed on a rotating flat platform and spun to produce thin films. Pressing, however, provides an alternative that allows for improved control of film thickness and offers the possibility of mixing by use of repeated pressing. This technique is similar to the approach used in droplet-array sandwiches. This effect is driven by the high cohesion of aqueous solvents and such an approach can be readily implemented into a lab-on-a-chip workflow.

### Sample Preparation

Accordingly, disclosed herein, in certain embodiments, is a method for preparing a biological sample (e.g., a protein sample) in a thin film for a bioprocessing assay (e.g., an assay for isolation, purification, identification, and/or characterization of a biological sample, such as a protein). The method includes, in certain embodiments, preparing a biological sample by combining an enzyme of interest with a substrate (e.g., a polypeptide, protein, nucleic acid, sugar, or lipid) at a predetermined ratio to produce a substrate-enzyme mixture (e.g., a SES). Non-limiting examples of enzymes suitable for use in conjunction with the disclosed methods include a protease, a glycosylase, and a phosphatase, among others. In certain embodiments, the protease is a trypsin, chymotrypsin, immunoglobulin degrading enzyme (IgDE), immunoglobulin G-degrading enzyme of Streptococcus pyogenes (IdeS), Asp-N protease, or Lys-C protease. In certain embodiments, the glycosylase is peptide:N-glycosidase F (PNGase F). In certain embodiments, the phosphatase is alkaline phosphatase (ALP). The sample is prepared, in certain embodiments, by mixing the enzyme and the substrate in an aqueous solvent (e.g., phosphate buffered saline (PBS), TRIS buffer, ammonium bicarbonate, sodium chloride, sodium sulfate, and the like) using known methods. The predetermined ratio of the enzyme and substrate is about 1:20, 1:25, 1:30, 1:40, 1:50, 1:60, 1:70, 1:80, 1:90, 1:100, 1:110, 1:120, 1:130, 1:140, 1:150, 1:160, 1:170, 1:180, 1:190, or 1:200 (mol enzyme:mol substrate), in certain embodiments. The substrate-enzyme mixture can be included in an aqueous solvent at various concentrations, including but not limited to between 0.1 and 20 mg/mL (e.g., 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, and 20 mg/mL). The total volume of the aqueous solvent containing the substrate-enzyme mixture is, in certain embodiments, between 10 and 100 μL (10 to 100 μL, 15 to 95 μL, 20 to 90 μL, 25 to 85 μL, 30 to 80 μL, 35 to 75 μL, 40-70 μL, 45 to 65 μL, and 50 to 60 μL). In certain embodiments, the substrate-enzyme mixture is prepared by, e.g., mixing 12.5 μL of a solution comprising the substrate at a concentration of 2 mg/mL with 12.5 μL of a solution comprising the enzyme at a concentration of 0.1 mg/mL. In certain embodiments, the substrate-enzyme mixture further comprises CaCl₂ (e.g., 4 mM), a surfactant (e.g., RAPIGEST™, e.g., 0.1%-0.5%), and guanidium hydrochloride.

In certain embodiments, the viscosity of the sample containing the substrate-enzyme mixture and solvent is adjusted using known methods to achieve a desirable consistency. For example, the solution is adjusted to have a viscosity of 0.1 to 2.0 mPa-S (e.g., 0.1 to 2.0 mPa-S, 0.2 to 1.9 mPa-S, 0.3 to 1.8 mPa-S, 0.4 to 1.7 mPa-S, 0.5 to 1.6 mPa-S, 0.6 to 1.5 mPa-S, 0.7 to 1.4 mPa-S, 0.8 to 1.3 mPa-S, 0.9 to 1.2 mPa-S, and 1.0 to 1.1 mPa-S), in certain embodiments. Various other parameters of the sample may be optimized to achieve advantageous properties, such as pH, temperature, homogeneity, melting point, boiling point, and saturation of the sample using routine methods.

### Thin Film Production

Once the sample containing the substrate-enzyme mixture is prepared, it is deposited on a conical or frustoconical surface, such as a conical or frustoconical surface of an apparatus disclosed herein, in a predetermined amount (e.g., between 10 and 100 μL), in certain embodiments. The sample containing the mixture is deposited on the surface using a variety of conventional methods and devices, such as, e.g., using a sample injection device operably connected to an apparatus containing two nested conical or frustoconical surfaces. Formation of a thin film containing the substrate-enzyme mixture proceeds by pressing the mixture between a bottom conical or frustoconical surface in which the sample is deposited with a top conical or frustoconical surface by applying a predetermined force between the two surfaces. In certain embodiments, the predetermined force is between 3 and 23 pounds (e.g., 3 to 20 lbs, 4 to 19 lbs, 5 to 18 lbs, 6 to 17 lbs, 7 to 16 lbs, 8 to 15 lbs, 9 to 14 lbs, 10 to 13 lbs, and 11 to 12 lbs). In certain embodiments, the predetermined force is between 10 and 15 pounds (e.g., 10 to 15 lbs, 11 to 14 lbs, and 12 to 13 lbs). Pressing of the top and bottom surface produces a nested conical or frustoconical interface in which the mixture is molded and which, in part, determines the thickness of the resulting thin film. In certain embodiments, pressing is performed once and for a predetermined duration (e.g., static pressing). In certain embodiments, the force is applied for a duration between 3 and 5 s (e.g., 3.0 to 5.0 s, 3.1 to 4.9 s, 3.2 to 4.8 s, 3.3 to 4.7 s, 3.4 to 4.6 s, 3.5 to 4.5 s, 3.6 to 4.4 s, 3.7 to 4.3 s, 3.8 to 4.2 s, and 3.9 to 4.1 s). Repeated pressing (e.g., active dynamic pressing) of the top and bottom surfaces for a predetermined duration can also be performed to facilitate mixing of the enzyme and substrate. Such dynamic pressing can include application and withdrawal of force at predetermined intervals and for a predetermined duration. For example, in certain embodiments, the application of force for a duration of 3 to 5 seconds is followed by withdrawal of force for a duration of between 1 and 2 s (e.g., 1.0 to 2.0 s, 1.1 to 1.9 s. 1.2 to 1.8 s, 1.3 to 1.7 s, and 1.4 to 1.6 s). In the active dynamic pressing regime, the application and withdrawal of force is performed over a total duration of about 30 s, in certain embodiments. In certain embodiments, the pressing step (e.g., static or dynamic pressing) is performed for a time sufficient to produce a thin film having a desirable thickness, density, consistency, compactness, viscidity, or acceleration of reaction rate of the SES. In certain embodiments, the thin film has a thickness no greater than about 100 μm, about 90 μm, about 80 μm, about 70 μm, about 60 μm, about 50 μm, about 40 μm, about 30 μm, about 20 μm, or about 10 μm. In certain embodiments, the thin film has a thickness no greater than about 50 μm.

In certain embodiments, the thin film accelerates a reaction rate of an SES by increasing the rate of binding of the enzyme to the substrate (Kₒₙ). In certain embodiments, the thin film accelerates the reaction rate of the SES by at least 1.5-fold, 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, 10,000-fold, or more, as compared to the reaction rate of the SES in a control bulk solution. In certain embodiments, the control bulk solution is of the same volume as the starting volume of the aqueous sample used to prepare the thin film. In certain embodiments, the control bulk solution is of the same temperature as the starting temperature of the aqueous sample used to prepare the thin film and/or the temperature of the thin film. In certain embodiments, the reaction of the SES in a thin film proceeds for the same duration as the reaction of the SES in a bulk control solution. In certain embodiments, the reaction of the SES (e.g., in the thin film or in the control bulk solution) is terminated using chemical means (e.g., guanidine hydrochloride).

### Apparatus for Production of Thin Films

Disclosed herein, in certain embodiments, is an apparatus configured to produce thin films containing a substrate-enzyme system (SES). Such an apparatus includes, in certain embodiments, a means for receiving an aqueous sample (e.g., a fluid inlet port), a means for holding the sample (e.g., a bottom conical or frustoconical surface), a means for controlling the temperature of the sample on the surface (e.g., a thermometer, thermoregulator, and heating elements), and a means for pressing the sample (e.g., a top conical or frustoconical surface operably connected to a linear actuator configured to produce a predetermined force). In certain embodiments, the means for receiving the aqueous sample, the means for holding the sample, the means for controlling the temperature of the sample, and the means for pressing the sample are operably connected to one another. In certain embodiments, the means for receiving an aqueous sample is physically connected to the means for holding the sample. In certain embodiments, the means for holding the sample is physically connected to the means for controlling the temperature of the sample. In certain embodiments, the means for pressing the sample is physically connected to the linear actuator.

### Top and Bottom Base Members

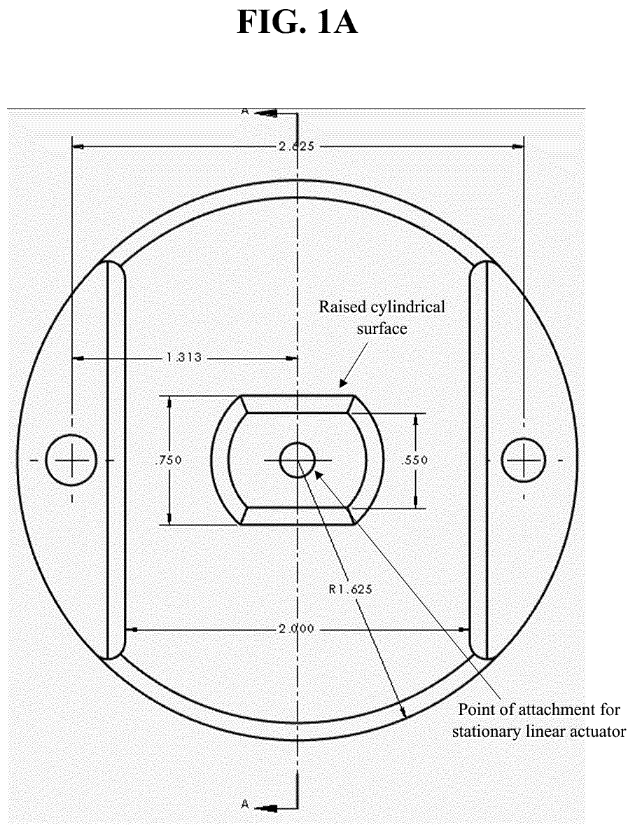

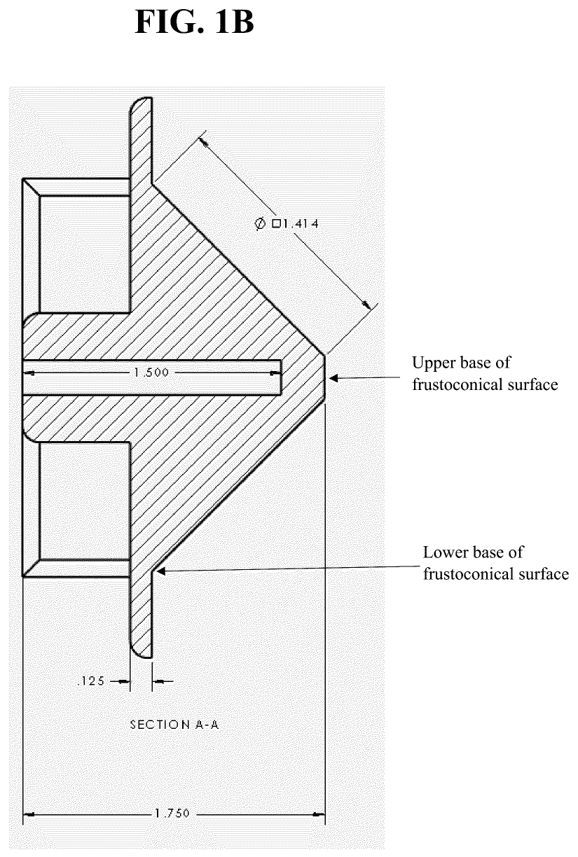

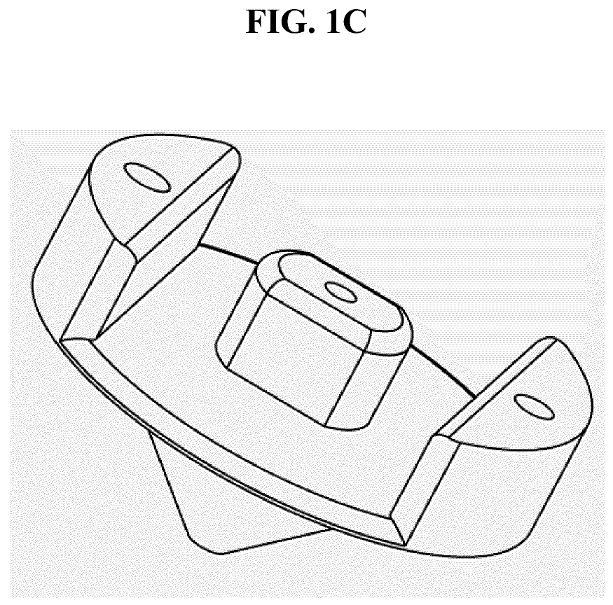

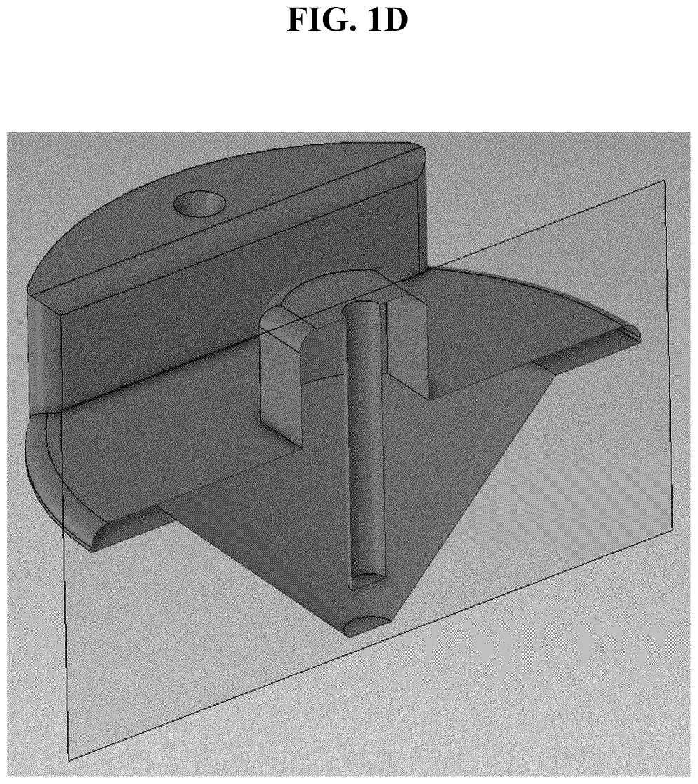

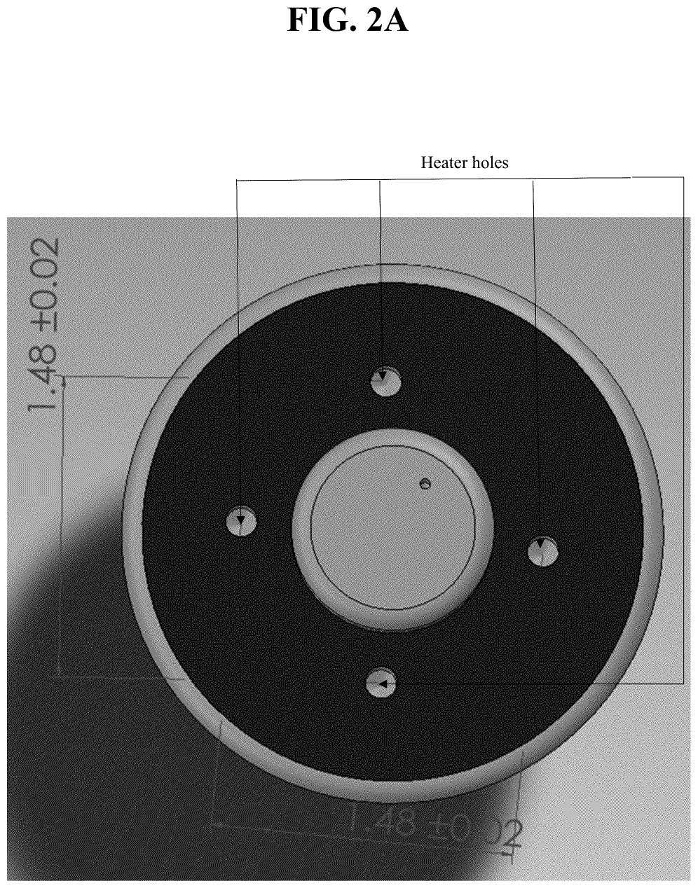

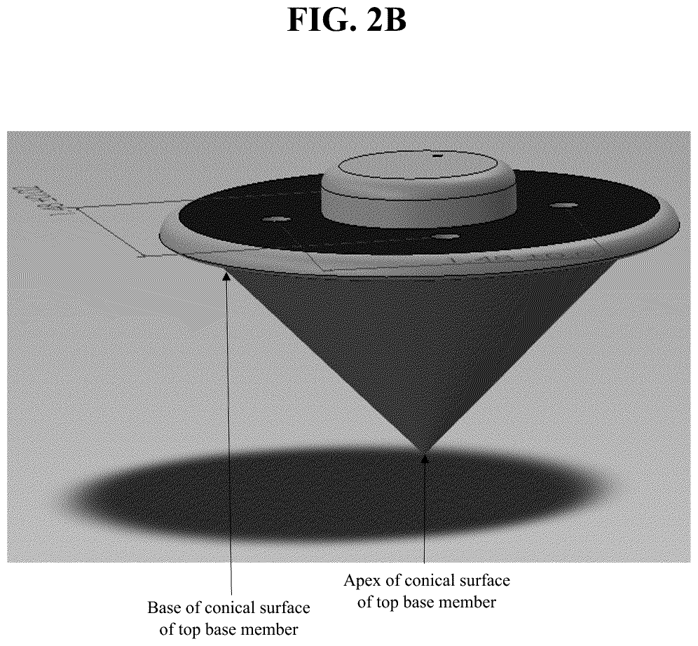

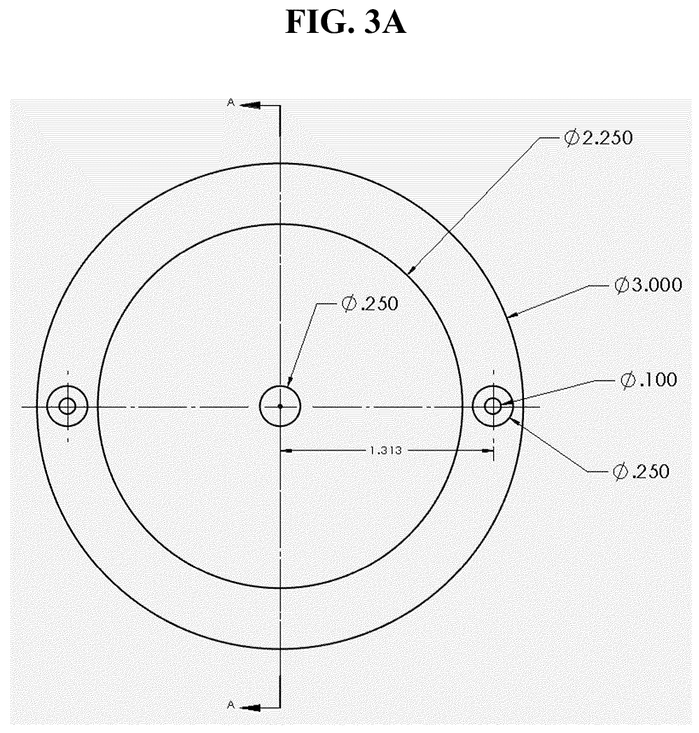

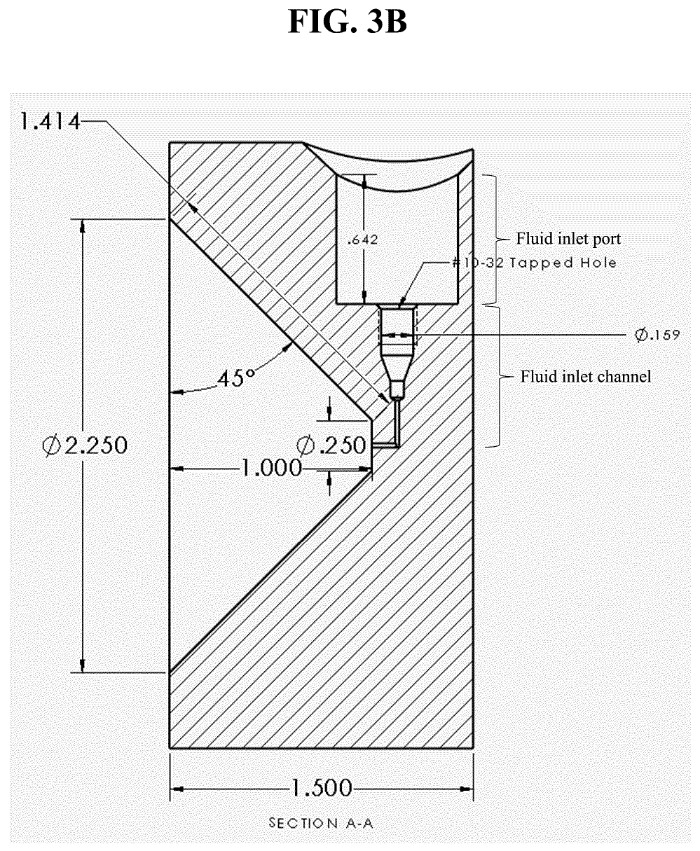

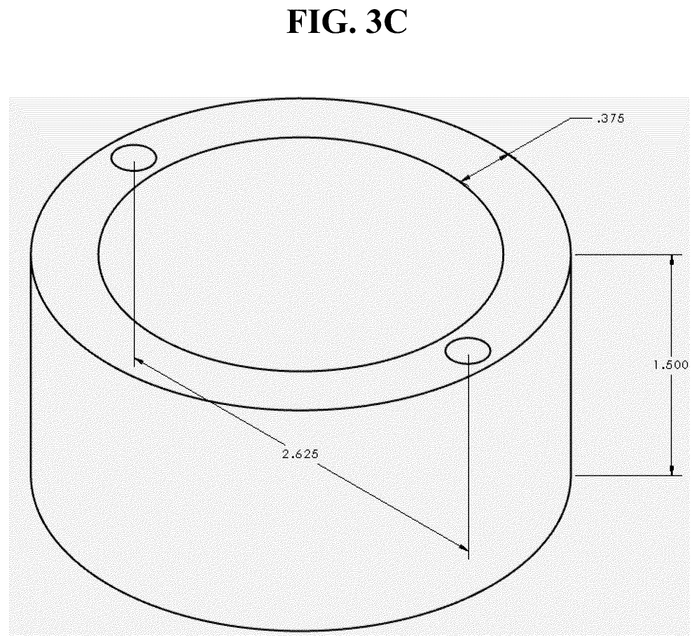

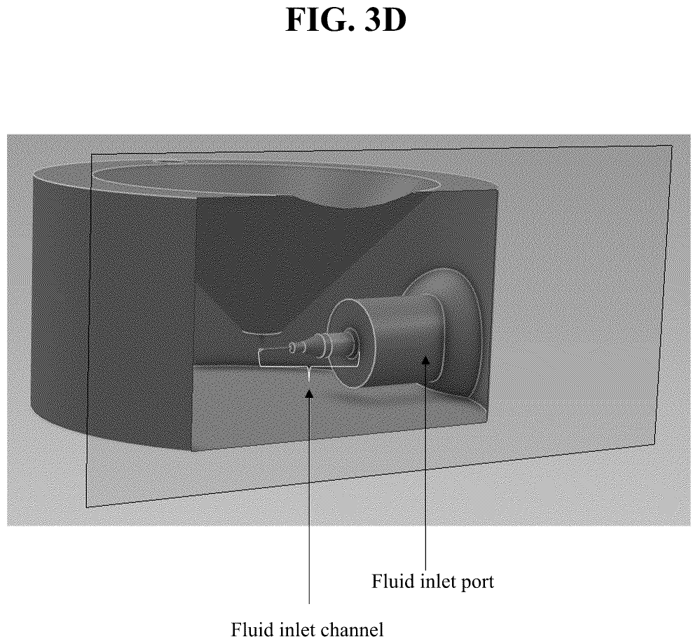

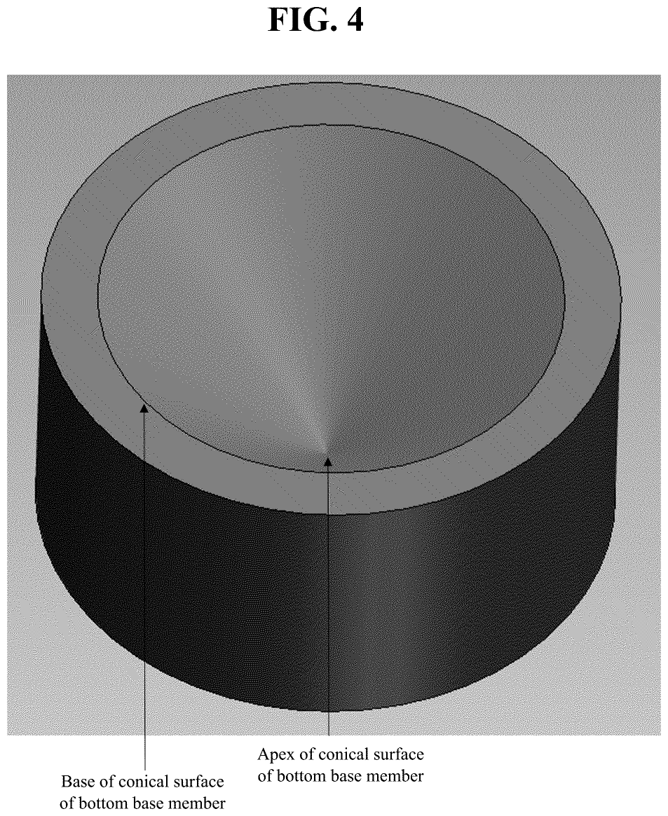

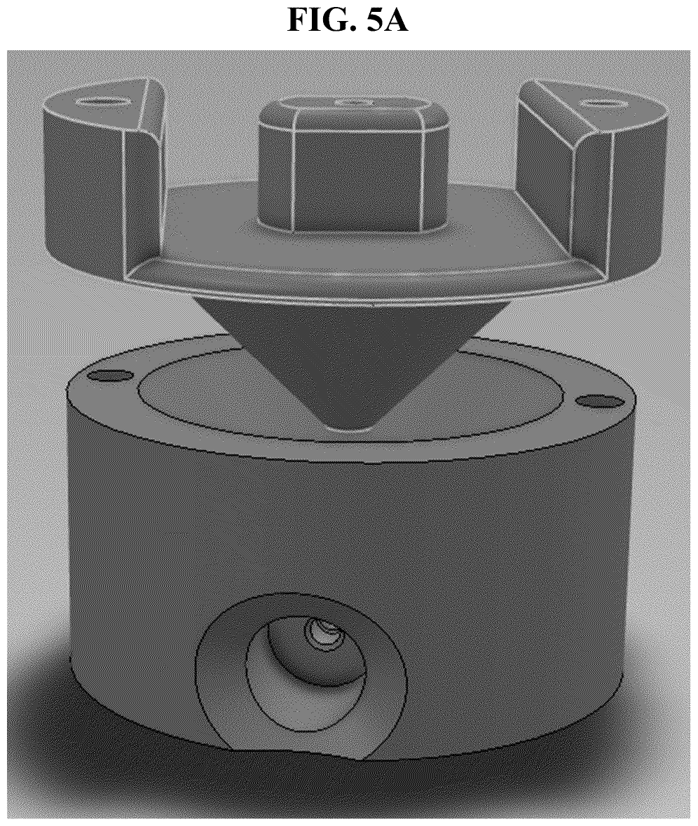

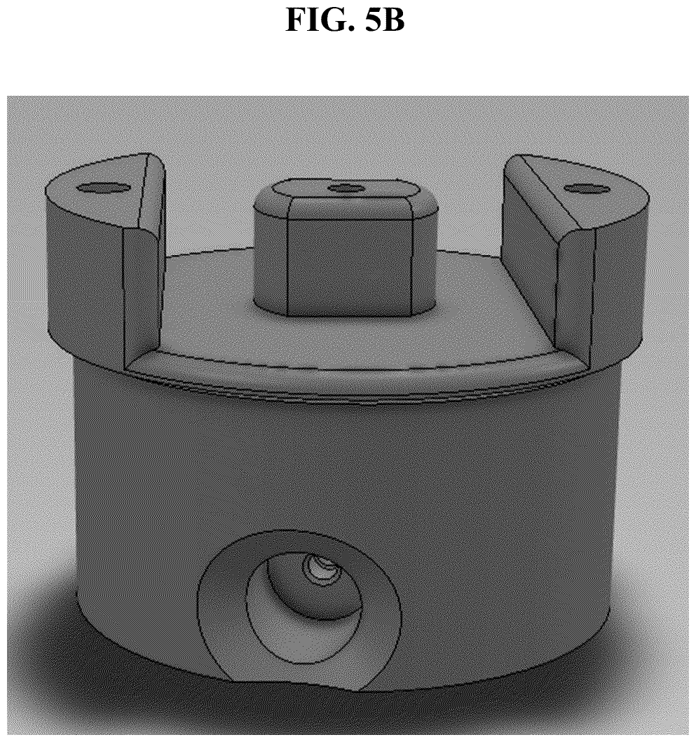

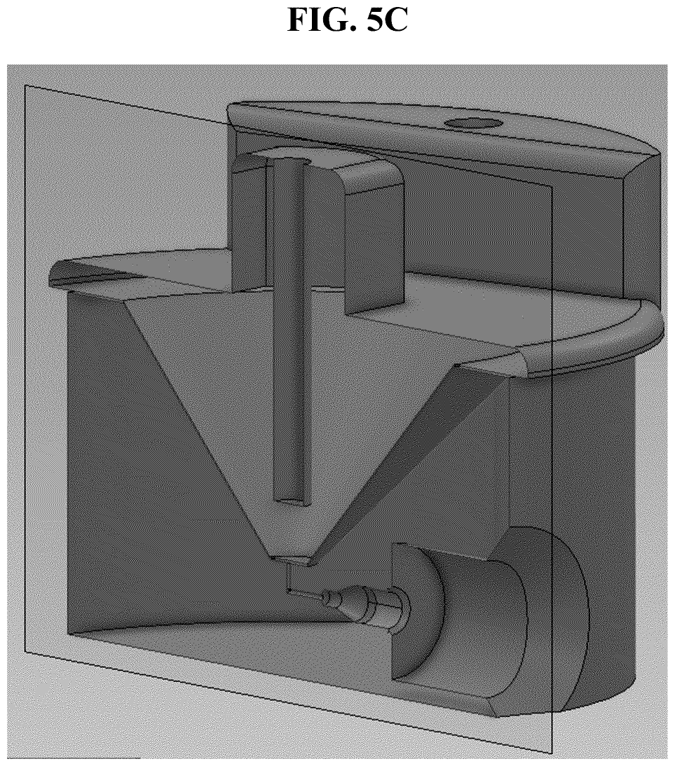

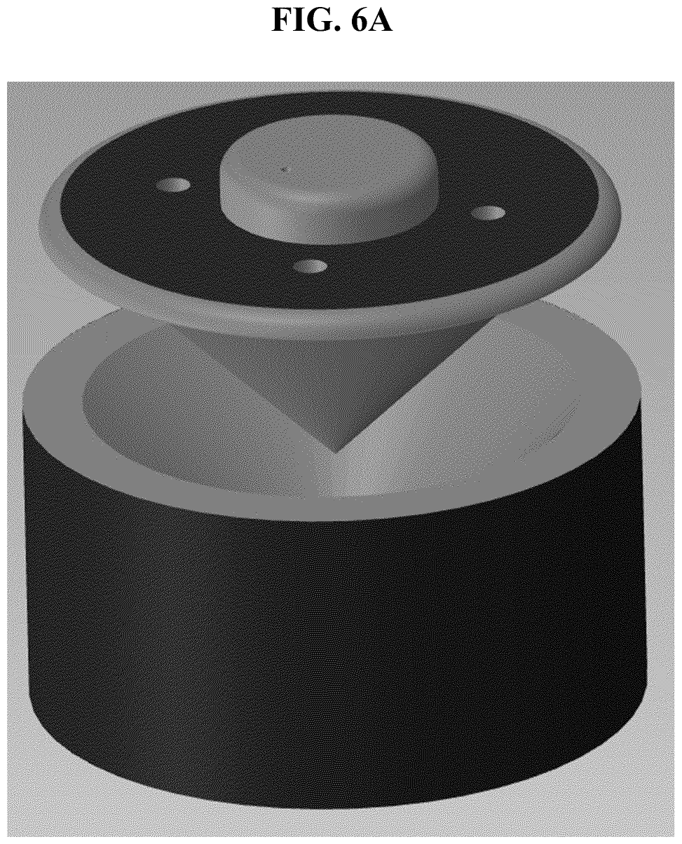

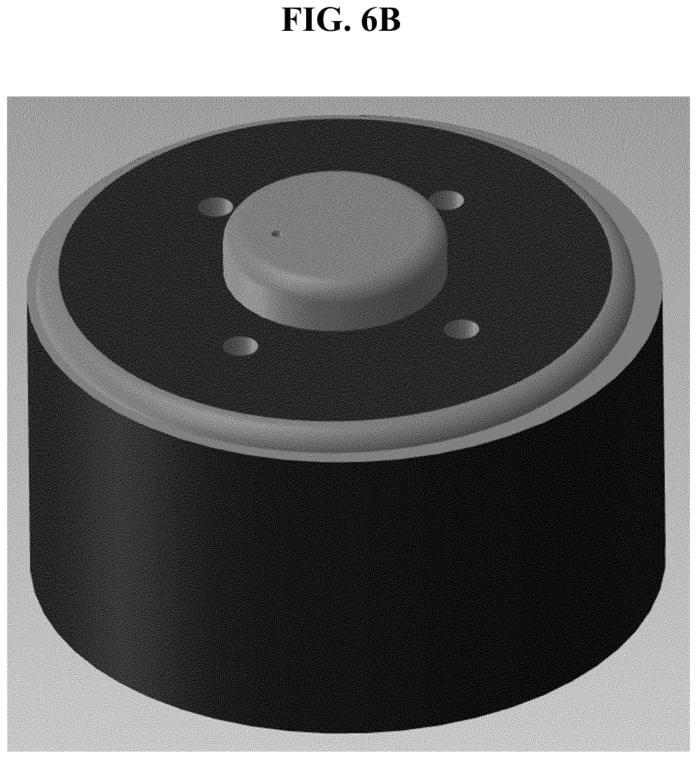

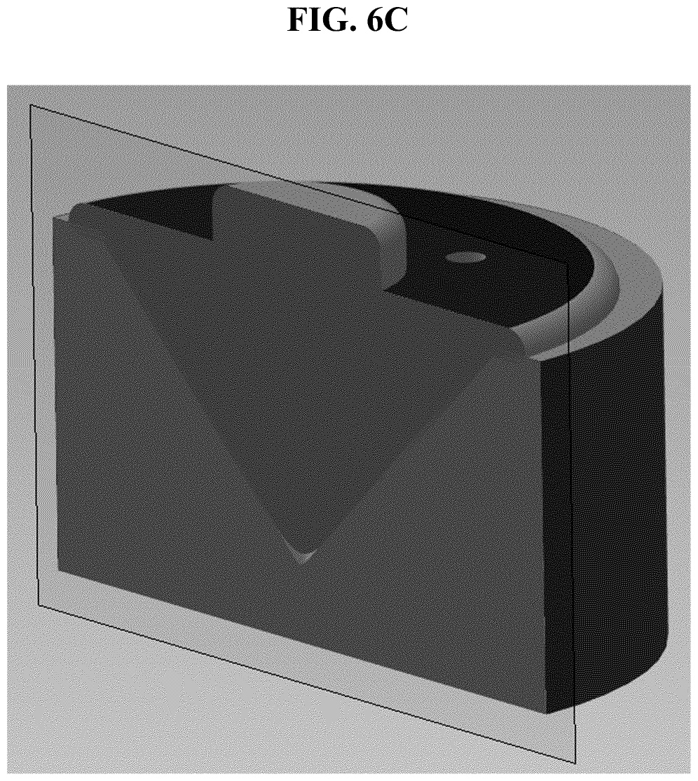

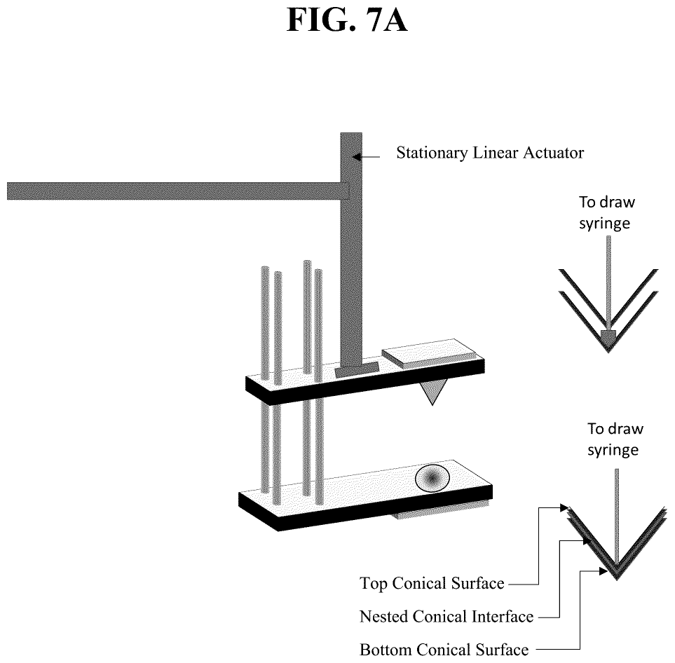

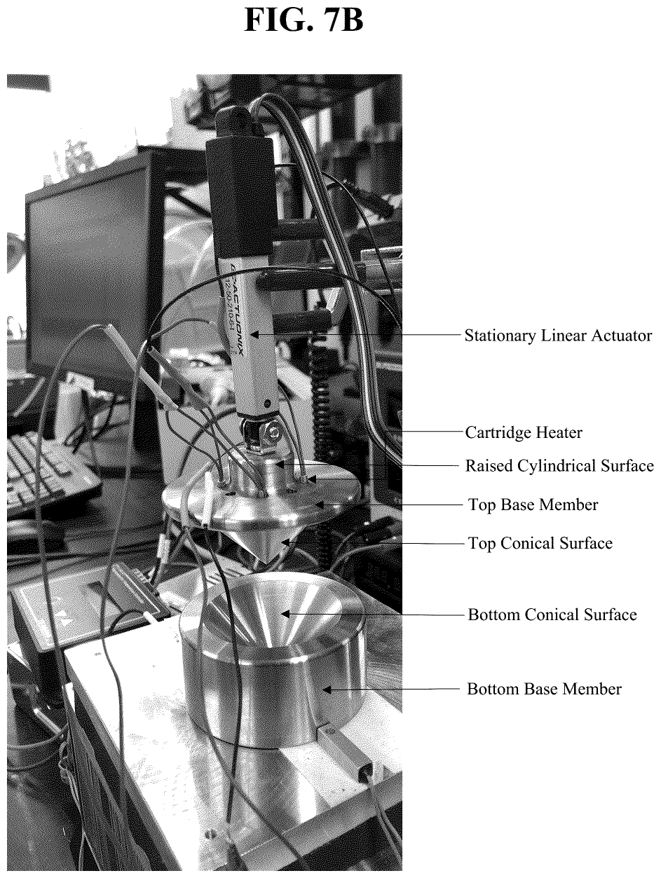

An apparatus configured to produce thin films containing an SES according to the present disclosure comprises a top base member (see, e.g., FIGS. 1A-D, FIGS. 2A-2B, and FIGS. 5A-5C, FIGS. 6A-6C, and FIG. 7B) and a bottom base member (see, e.g., FIGS. 3A-D, FIG. 4, FIGS. 5A-5C, FIGS. 6A-6C, and FIG. 7B) having a conical (see, e.g., FIGS. 2A-2B and FIG. 4) or a frustoconical surface (see, e.g., FIGS. 1A-1D and FIGS. 3A-3D), in certain embodiments. The top base member includes, on one side, a vertically-oriented, downward-facing, and convex conical (FIG. 2B and FIG. 7B) or frustoconical surface (FIGS. 1B-1D), said conical or frustoconical surface being attached to the top base member at the base of the conical surface or the bottom base of the frustoconical surface (see, e.g., FIGS. 1A-D and FIGS. 2A-2B). On the other, upward-facing side of the top base member is a raised cylindrical surface on the top base member (see, e.g., FIGS. 1A-D, FIGS. 2A-2B, FIGS. 5A-5C, FIGS. 6A-6C, and FIG. 7B) configured to operably connect to a stationary linear actuator which is configured to move along the vertical axis (FIGS. 7A-7B), in certain embodiments. The bottom base member is, in certain embodiments, a cylinder with a hollowed out, vertically-oriented, and concave conical (FIG. 4, and FIGS. 6A and 6C) or frustoconical surface. In certain embodiments, the bottom base member is operably connected to a stationary linear actuator which is configured to move along the vertical axis and bring the top and bottom conical or frustoconical surfaces in contact or sufficiently close proximity to produce a thin film. In certain embodiments, both the top and the bottom base members are operably connected to a stationary linear actuator. In certain embodiments, only the top base member is operably connected to a stationary linear actuator. In certain embodiments, only the bottom base member is operably connected to the stationary linear actuator. Although the size of the apparatus may be scaled arbitrarily, in certain embodiments, the top and/or bottom conical or frustoconical surface of the apparatus may each have a slant height of 3 to 15 cm (e.g., 3, 4, 5, 6, 7, 8, 9, 10, 11, 12, 13, 14, and 15 cm). In certain embodiments, the top conical or frustoconical surface and the bottom conical or frustoconical surface each have a vertical angle of 45° . For cases in which a frustoconical surface is used in lieu of a conical surface, the top frustoconical surface and the bottom frustoconical surface comprise an upper base having a diameter of 1 mm to 10 mm (e.g., 1, 2, 3, 4, 5, 6, 7, 8, 9, and 10 cm). With respect to the fluid sample used for producing a thin film of the disclosure, the bottom conical or frustoconical surface is configured to hold a fluid volume of at least between 10 μL and 100 μL (e.g., 10 to 100 μL, 15 to 95 μL, 20 to 90 μL, 25 to 85 μL, 30 to 80 μL, 35 to 75 μL, 40 to 70 μL, 45 to 65 μL, and 50 to 60 μL).

### Sample Delivery

As discussed above, the apparatus of the disclosure includes a means for receiving an aqueous sample, such as a fluid inlet port operably connected to the top or bottom conical or frustoconical surface, in certain embodiments. In certain embodiments, the fluid inlet port is operably connected to the bottom conical or frustoconical surface. In certain embodiments, the fluid inlet port is operably connected to the top conical or frustoconical surface. The fluid inlet port may be of any configuration, provided it is capable of supplying a fluid sample (e.g., a fluid sample comprising the SES) to the top or bottom base member in a predetermined volume, for a predetermined time, and/or at a predetermined temperature. In certain embodiments, the fluid inlet port is operably connected, e.g., by tubing, to a fluid source containing the SES in an aqueous solvent and, optionally, operably connected to a fluid pump configured to move the fluid sample from the fluid source to the fluid inlet port. The fluid inlet port directs and deposits the fluid sample from the fluid source to the bottom conical or frustoconical surface via a fluid inlet channel, in certain embodiments. In certain embodiments, the fluid inlet channel is operably connected to the top or bottom conical or frustoconical surface. Regardless of whether the fluid inlet channel is connected to the top or bottom surface, the action of gravity assists the deposition of the fluid sample on the bottom conical or frustoconical surface, which serves as a means for holding the fluid sample prior to pressing and, optionally, mixing of the fluid sample.

### Pressing and Mixing

The apparatus of the disclosure includes, in certain embodiments, a means for pressing the fluid sample so as to produce a pressed, and optionally mixed, thin film from said sample. In certain embodiments, the means for pressing the fluid sample is a stationary linear actuator operably connected to a top or bottom base member, or both. The linear actuator moves the top or bottom base members, or both, along a vertical axis such that the top conical or frustoconical surface of the top base member and the bottom conical or frustoconical surface of the bottom base member come into contact or close proximity (e.g., within 1-30 μm of one another) to create a nested conical or frustoconical interface in which the fluid sample is physically pressed to produce a thin film comprising the SES. In certain embodiments, the linear actuator is configured to generate a predetermined force between the top conical or frustoconical surface and the bottom conical or frustoconical surface that is between 3 and 20 pounds e.g., 3 to 20 lbs, 4 to 19 lbs, 5 to 18 lbs, 6 to 17 lbs, 7 to 16 lbs, 8 to 15 lbs, 9 to 14 lbs, 10 to 13 lbs, and 11 to 12 lbs). Additionally, the linear actuator might be vertically positioned with a slight offset angle (e.g.,) 10°-30° such that the actuator produces slight lateral (i.e., horizontal) motion to assist in reducing the surface tension of the film.

### Temperature Control

As discussed above, the disclosed apparatus includes, in certain embodiments, a means for controlling the temperature of the sample (e.g., fluid sample or thin film) on one or more surfaces of the apparatus. Accordingly, temperature control of the apparatus may be instantiated by use of, e.g., one or more (e.g., 1, 2, 3, 4, or more) heating elements (e.g., cartridge heaters or an infrared laser) operably connected to the top or bottom base member. In certain embodiments, the one or more heating elements are operably connected to one or more thermistors, relay circuits, and a thermoregulator. The temperature range suitable for preparation of thin films of the disclosure may vary depending on the specific SES preparation, but can include temperatures between 20° C.-80° C.

### Surface Properties

According to the present disclosure, the surfaces (e.g., top and bottom conical or frustoconical surfaces) of the apparatus disclosed herein may be of any material known to be suitable for the production of thin films. For example, the surfaces may be made from stainless steel, poly(methyl acrylate), or glass, among others.

Additionally, the top and/or bottom conical or frustoconical surface of the apparatus disclosed herein may be modified by physical or chemical means in order to obtain optimal surface properties for the preparation of thin films as disclosed herein. For example, the top and/or bottom conical or frustoconical surface may be physically modified by deposition-based coating with, e.g., polyethylene glycol (PEG), phenyl, or C2. Additionally or alternatively, the top and/or bottom conical or frustoconical surface may be functionalized by chemical means, e.g., by silanolization followed by functionalizing with PEG, phenyl, C2, or other modifiers.

### Bioprocessing Assays

The methods and devices disclosed herein are used in for biological sample preparation in a variety of bioprocessing assays, in certain embodiments. As discussed above, the time required for preparative reactions for analytical techniques in the bioprocessing space can be prohibitive to efficient workflows by introducing delays stemming from limitations in reaction rates. These delays impede access to real-time results needed in feedback loop-modulated bioreactor design. Accordingly, the disclosed methods and devices provide improvements in sample preparation for bioprocessing/analytical assays by reducing rates of preparative reactions. Without limitation, the disclosed methods and devices are suitable for use in conjunction with a variety of bioprocessing assays, including peptide mapping, peptide monitoring, proteomics, protein purification, glycan characterization, glycomics, and glycoprotein characterization.

Analysis of the primary structure of proteins is necessary for the characterization of proteins. Peptide mapping (also known as peptide mass fingerprinting) is a valuable approach to combine positional quantitative information with topographical and domain information of proteins. In particular, peptide mapping is a useful procedure and a critical goal of many genome sequencing projects and biomedical and biopharmaceutical research efforts. In a typical peptide mapping workflow, fragmentation of isolated proteins by enzymatic digestion (e.g., with a protease, such as trypsin, chymotrypsin, pepsin, glutamyl endopeptidase, etc.) is performed followed by separation and analysis using methods such as mass spectrometry (MS) or high-performance liquid chromatography (HPLC). Masses of digested peptide fragments are then compared to databases containing reference protein sequences and their corresponding fragments. Comparison of the mass of fragments of the unknown protein to known protein fragments aids in the identification and characterization of the unknown protein. Therefore, the disclosed methods and devices are suitable for use in conjunction with peptide mapping techniques.

Relatedly, peptide monitoring if often performed for analysis of quality of biotherapeutic drugs (e.g., peptide or protein therapeutics). Proteolytic digests are often analyzed as a monitoring technique for protein composition of biotherapeutic drugs. The high costs associated with meeting regulatory guidelines for biotherapeutic drug safety has renewed interest in efficient methods to reduce costs and increase productivity in the manufacturing process.

Furthermore, proteomic analysis often involves analysis of proteins using electrophoretic separation, MS, and/or liquid chromatography methods. In various applications of these methods, protein fragmentation and labeling are performed on proteins in a complex mixture, following by chromatographic separation and MS analysis of labeled peptide fragments. The efficiency and throughput of such analysis is dependent, at least in part, on the efficiency of protein preparation (e.g., enzymatic digestion of a protein sample). Accordingly, the disclosed methods and devices may be used in conjunction with proteomic analysis to improve its efficiency and cost.

Protein purification is yet another method performed on proteins for the purpose of isolation of a protein(s) of interest from a complex biological sample (e.g., protein mixture, cells, tissues, etc.) for, e.g., sample preparation and/or characterization of protein structure, function, quantity, etc. A typical protein purification and quantification workflow involves isolation of the protein of interest, denaturation of the tertiary and secondary structures of the protein for efficient digestion, and enzymatic cleavage of proteins at predicted amino acid residues to reduce sample complexity, thereby improving sensitivity and specificity of proteomic analysis.

Characterizing and monitoring the glycan population of a biotherapeutic protein (e.g., glycoprotein profiling, released glycan characterization and monitoring, subunit analysis, monosaccharide/sialic acid composition analysis, and glycopeptide mapping) has continued to present technical and logistical challenges to the biopharmaceutical industry due, at least in part, to limitations in efficiency and productivity of preparative reactions containing glycoproteins. As in proteomic analysis, glycan and glycoprotein sample analysis commonly involves preparative digestion of glycoproteins for their subsequent analysis and characterization, which can become a bottleneck when preparative reactions are rate-limiting. Accordingly, the disclosed methods and devices can be advantageously used with glycan and glycoprotein characterization and monitoring in order to improve efficiency of such analysis.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention

### Example 1: Tryptic Digestion of Bovine Fibrinogen Using a Pressed Thin Film

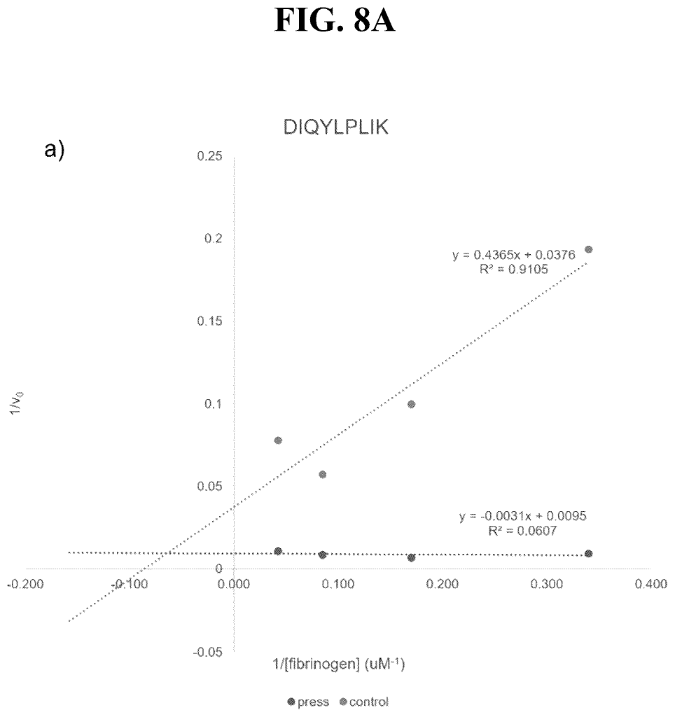

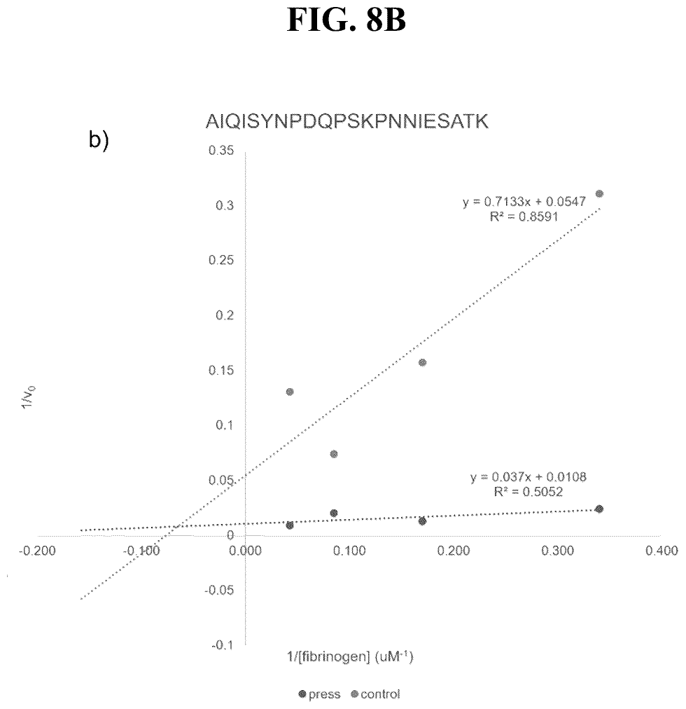

To assess the enzymatic digestion efficacy of substrate-enzyme systems (SES) prepared on a pressed thin film of the disclosure, the following steps were performed. Bovine fibrinogen was digested using bulk phase or isothermal thin film digestion. Trypsin enzyme was prepared at 0.1 mg/mL in 100 mM ammonium bicarbonate with 4 mM calcium chloride. Bovine fibrinogen was prepared at 1, 2, 4, and 8 mg/mL in 100 mM ammonium bicarbonate with 0.2% RAPIGEST™ surfactant. The reaction was initiated by mixture of 12.5 μL of each solution and quenched by the addition of 200 μL of guanidine hydrochloride. Peptide abundances were determined by liquid chromatography-mass spectrometry (LC-MS) of the resultant mixture. The thin film was generated using an apparatus as disclosed herein. The inverse of the initial reaction velocity, V₀, for peptides DIQYLPLIK (SEQ ID NO: 1) and AIQISYNPDQPSKPNNIESATK (SEQ ID NO: 2) were plotted against inverse fibrinogen concentration, yielding a Lineweaver-Burke plot (FIGS. 8A and 8B). The x-intercept of the Lineweaver-Burke plot equals −1/Kₘ, where Kₘ is the Michaelis constant; the y-intercept corresponds to 1/Vₘₐₓ, where Vₘₐₓ is the maximum rate of an enzyme-catalyzed reaction; and the slope of the plot is proportional to Kₘ/Vₘₐₓ. These plots demonstrate the independence of V₀ on substrate concentration in the thin film and show that for certain peptides, the Vₘₐₓ of the reaction is substantially increased with the use of a thin film preparation as compared to a control bulk solution. The Vₘₐₓ calculated for DIQYLPLIK (SEQ ID NO: 1) in the thin film was 11.1 counts/s, as compared to 2.2 counts/s in bulk solution. The Vₘₐₓ for AIQISYNPDQPSKPNNIESATK (SEQ ID NO: 2) was 12.6 counts/s in the thin film as compared to 0.3 counts/s in bulk solution.

### Example 2: Tryptic Digestion of a Monoclonal Antibody Using a Pressed Thin Film

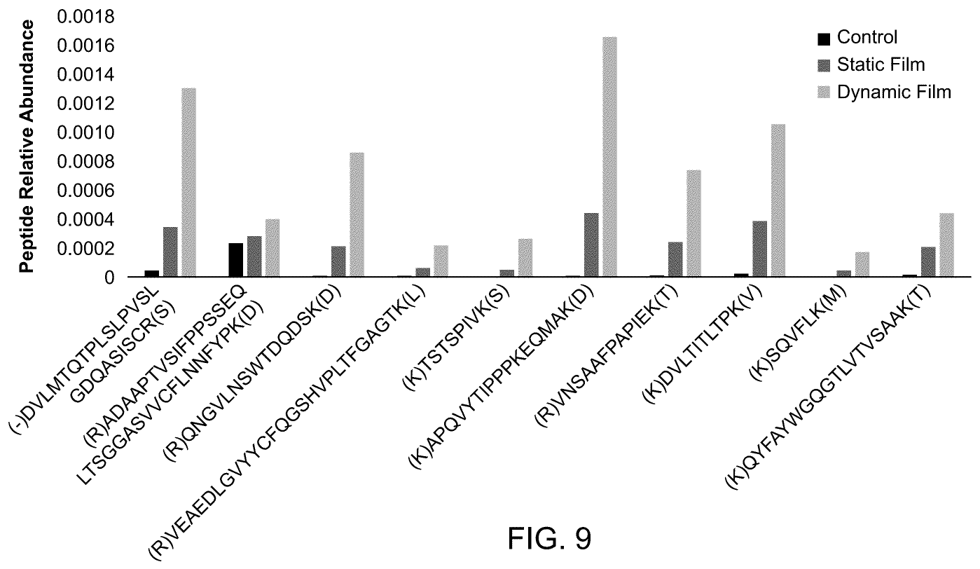

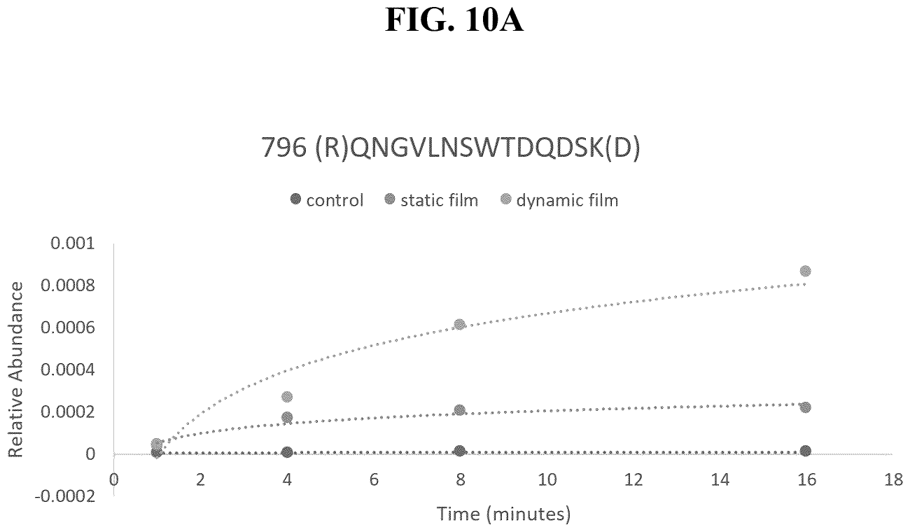

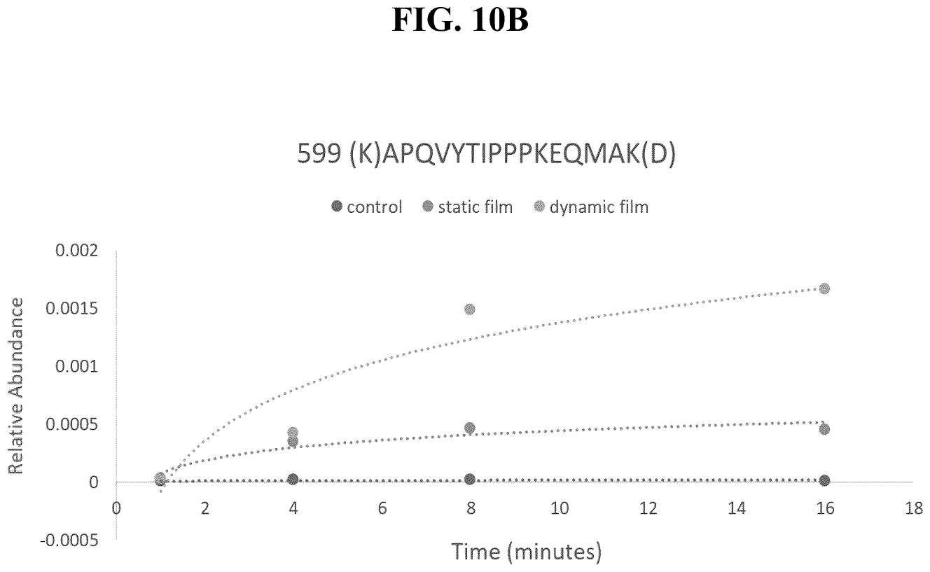

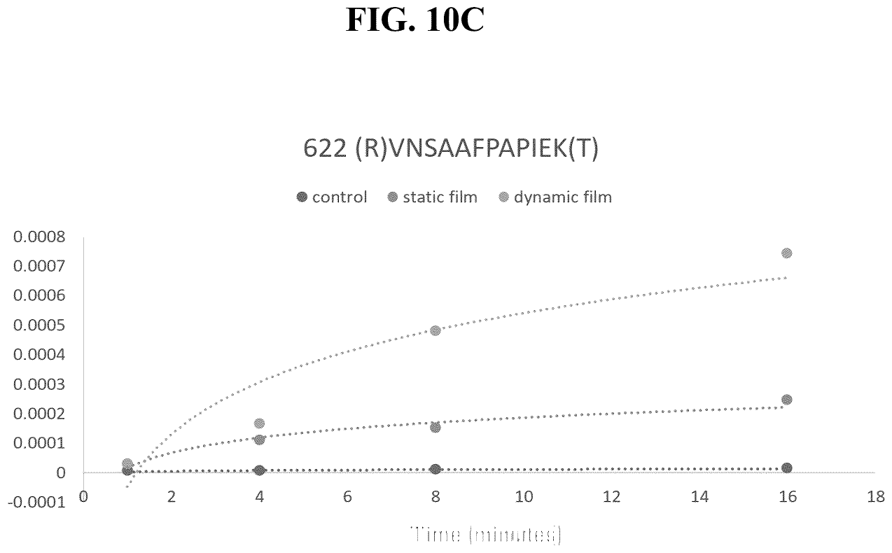

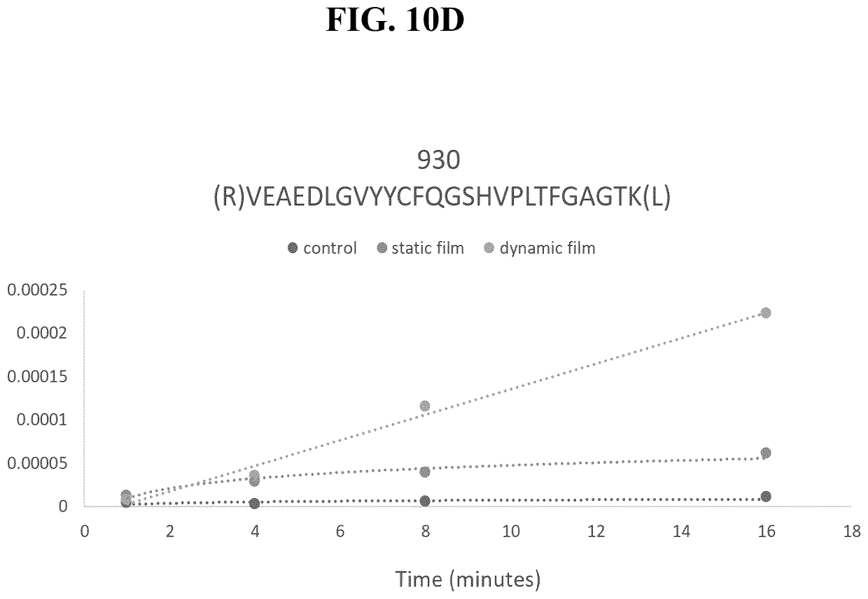

The Waters Intact Monoclonal Antibody Check Standard was prepared at 2 mg/mL in 100 mM ammonium bicarbonate with 0.2% RAPIGEST™ surfactant. Trypsin was prepared at 0.1 mg/mL in 100 mM ammonium bicarbonate with 4 mM calcium chloride. Enzymatic digestion was initiated by mixing of 12.5 μL of each solution and quenched with 200 μL of 6M guanidine hydrochloride. Peptide abundances were determined by LC-MS of the resultant mixture. Peptide abundance for multiple digested peptide fragments of the digested antibody using static or dynamic thin films or a control bulk solution over the course of 16 minutes. Tested peptide fragments included (−)DVLMTQTPLSLPVSLGDQASISCR(S) (SEQ ID NO: 3), (R)ADAAPTVSIFPPSSEQLTSGGASVVCFLNNFYPK(D) (SEQ ID NO: 4), (R)QNGVLNSWTDQDSK(D) (SEQ ID NO: 5), (R)VEAEDLGVYYCFQGSHVOKTFGAGGTK(L) (SEQ ID NO: 6), (K)TSTSPIVK(S) (SEQ ID NO: 7), (K)APQVYTIPPPKEQMAK(D) (SEQ ID NO: 8), (R)VNSAAFPAPIEK(T) (SEQ ID NO: 9), (K)DVLTITLTPK(V) (SEQ ID NO: 10), (K)SQVFLK(M) (SEQ ID NO: 11), and (K)QYFAYWGQGTLVTVSAAK(T) (SEQ ID NO: 12) (FIG. 9). Amino acid positions denoted by parentheses are not part of the peptide fragments, but correspond to preceding and succeeding amino acids from the full-length polypeptide from which they are generated (“(−)” denotes no preceding or succeeding amino acid at the position shown). In all cases, thin film reaction systems improved protein digestion efficacy as compared to the bulk solution reaction system. For certain peptide fragments, relative peptide abundance was substantially higher when using the dynamic mixing as compared to the static mixing (FIG. 9 and FIGS. 10A-10D).

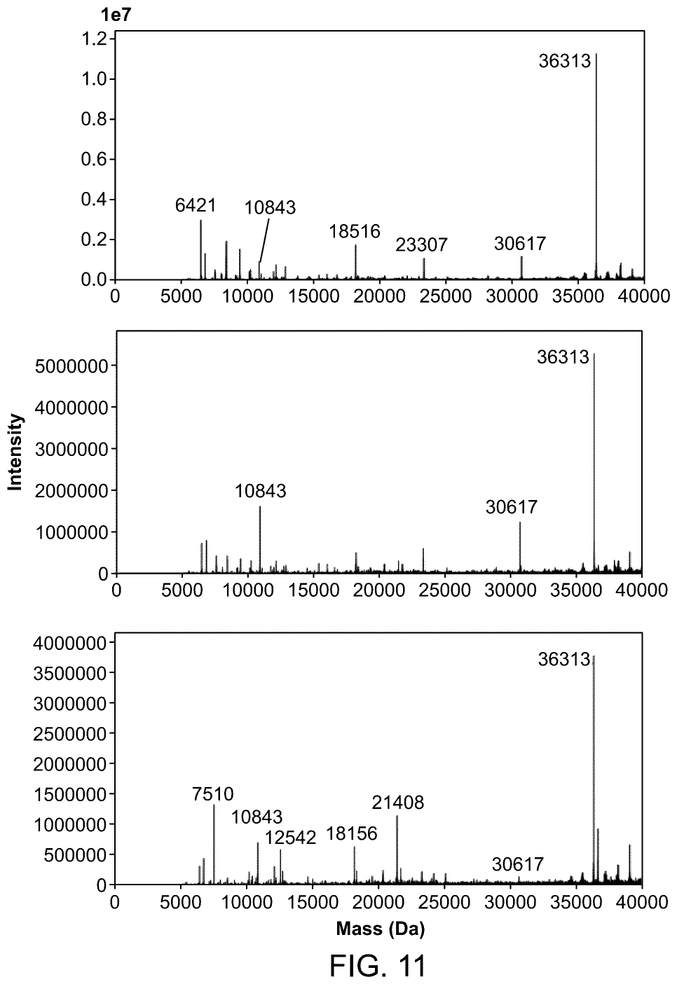

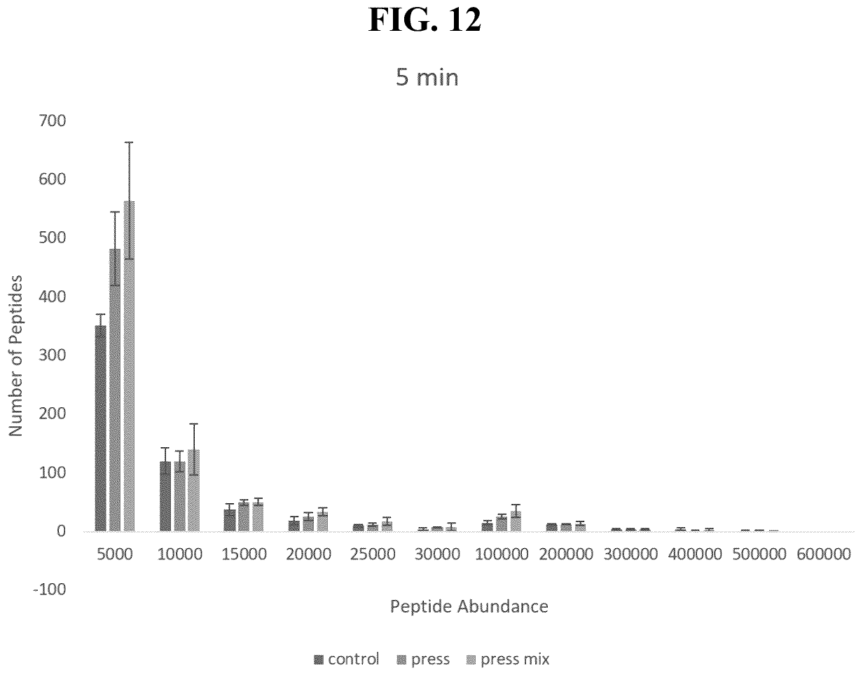

Analysis of the deconvolved mass spectrum of the digested antibody showed that abundance of the large 36 kDa fragment was notably reduced in the static and dynamic thin film digestions, as compared to bulk solution digestion. These digestions instead feature numerous smaller fragments in the 5-10 kDa range. This indicates improved digestion of the intact protein into smaller intermediates in the thin film system (FIG. 11), particularly those below 10,000 (peptide abundance) (FIG. 12).

### Example 3: Effects of Pressed Thin Film Thickness on Enzymatic Digestion of a Monoclonal Antibody

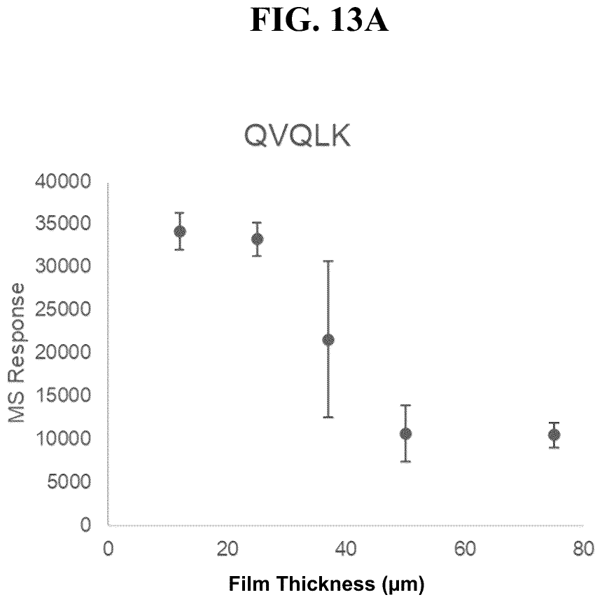

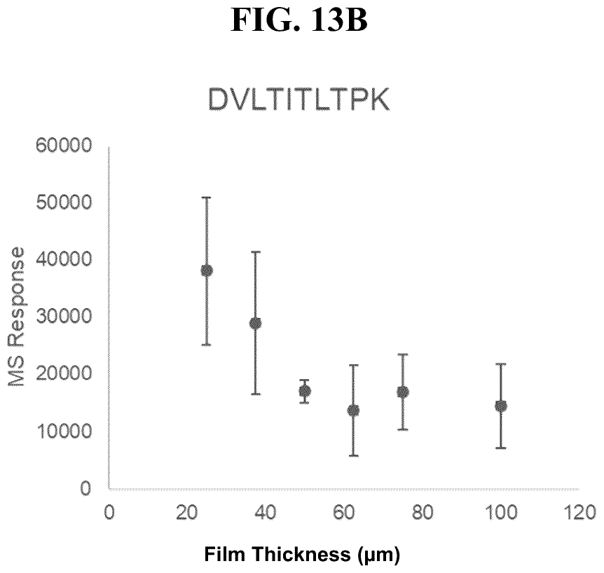

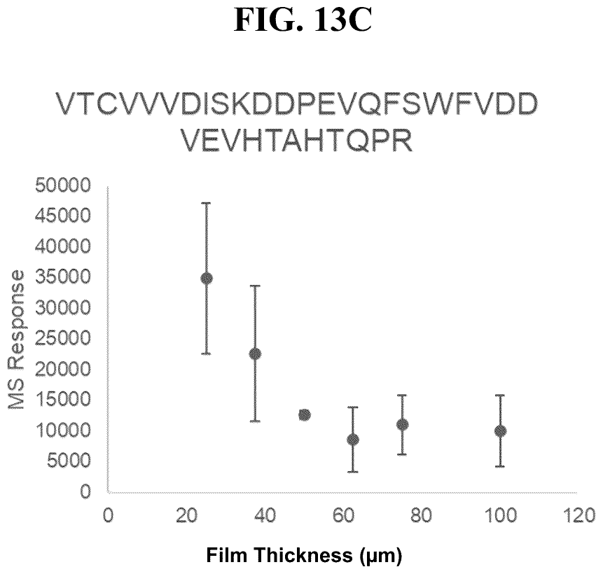

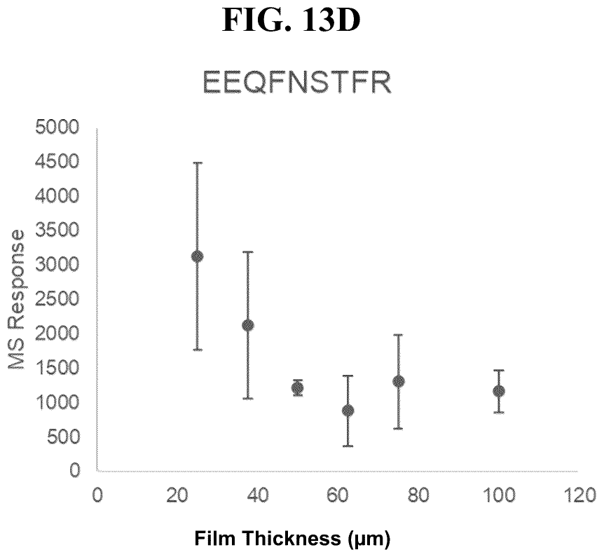

To determine the effect of pressed thin film thickness of enzymatic digestion of a reference monoclonal antibody (Waters Mass Check Standard), annular shims of inner and outer diameter equal to the rim of the bottom frustoconical plate were created in thicknesses of 12.5, 25, and 37.5 μm, such that stacking of the shims resulted in an offset gap being applied to the two plates. Reactions were conducted as one-pot digests in which 5 mM TCEP and enzyme were added to the antibody in 10 mM ammonium bicarbonate with 0.2% RAPIGEST™ detergent. The reaction was conducted at 55C.° for 20 minutes, and the resultant peptide abundance for the digested peptides was analyzed by LC-MS. The yield of four peptides, QVQLK (SEQ ID NO: 13), DVLTITLTPK (SEQ ID NO: 14), VTCVVVDISKDDPEVQFSWFVDDVEVHTAHTQPR (SEQ ID NO: 15), and EEQFNSTFR (SEQ ID NO: 16), as a function of film thickness is provided in FIGS. 13A-13D, demonstrating the effect of reducing film thickness on reaction yields. The thin film effect is evident for thicknesses less than 50 μm and produces a linear response with reaction yield.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A method of preparing a protein sample in a thin film for a bioprocessing assay, comprising:
(a) combining an enzyme and a protein substrate at a predetermined ratio to produce an substrate-enzyme mixture;
(b) depositing the mixture of step (a) in a bottom conical or frustoconical surface; and
(c) pressing the mixture between the bottom conical or frustoconical surface and a top conical or frustoconical surface by applying a predetermined force between the top conical or frustoconical surface and the bottom conical or frustoconical surface, wherein the top conical or frustoconical surface and bottom conical or frustoconical surface are vertically oriented and configured to produce a nested conical or frustoconical interface;
thereby forming a thin film comprising the protein-enzyme mixture in the nested conical interface.

2. The method of claim 1, wherein the predetermined ratio of the enzyme and protein substrate is about 1:20, 1:25, 1:30, 1:40, 1:50, 1:60, 1:70, 1:80, 1:90, 1:100, 1:110, 1:120, 1:130, 1:140, 1:150, 1:160, 1:170, 1:180, 1:190, or 1:200 (mol enzyme:mol substrate).

3. The method of claim 1, wherein the substrate-enzyme mixture is in a solution at a concentration of 0.1 to 20 mg/mL.

4. The method of claim 3, wherein the enzyme mixture is in 10 μL to 100 μL of the solution.

5. The method of claim 1, wherein the solution has a viscosity of 0.1 to 2.0 mPa-S.

6. The method of claim 1, wherein the predetermined force is between 3 and 20 pounds.

7. (canceled)

8. The method of claim 1, wherein the force is applied for a duration between 3 and 5 s.

9. The method of claim 8, wherein the application of force is followed by withdrawal of force for a duration of between 1 s and 2 s.

10. The method of claim 8, wherein the application and withdrawal of force is performed over a total duration of 30 s.

11-18. (canceled)

19. An apparatus for production of a thin film, comprising:
(a) a vertically-oriented, convex, top conical or frustoconical surface, wherein the base of the top conical or frustoconical surface is attached to a top base member;
(b) a vertically-oriented, concave, bottom conical or frustoconical surface configured to hold a sample volume, wherein the bottom conical or frustoconical surface is inside of a cylindrical bottom base member;
(c) a stationary linear actuator configured to move along a vertical axis and operably connected to a raised cylindrical surface on the top base member or operably connected to the bottom base member;
(d) a fluid inlet port operably connected to the top conical or frustoconical surface and/or the bottom conical or frustoconical surface;
(e) one or more heating elements operably connected to a temperature controller and relay circuit;
(f) a thermistor configured for placement inside a thermistor hole in the raised cylindrical surface of the top base member, said thermistor being operably connected to the temperature controller;
wherein the top conical or frustoconical surface and the bottom conical or frustoconical surface are configured to produce a nested conical or frustoconical interface.

20-23. (canceled)

24. The apparatus of claim 19, wherein the linear actuator is configured to generate a predetermined force between the top conical or frustoconical surface and the bottom conical or frustoconical surface that is between 3 and 20 pounds.

25. The apparatus of claim 19, wherein the linear actuator is configured to generate angular movement at an offset angle of 10°-30°.

26. The apparatus of claim 19, wherein the top and/or bottom conical or frustoconical surface each has a slant height of 3 cm to 15 cm.

27. The apparatus of claim 19, wherein the bottom conical or frustoconical surface has a vertical height of 2.85 cm to 10 cm.

28. The apparatus of claim 19, wherein the top conical or frustoconical surface and the bottom conical or frustoconical surface each have a vertical angle of 45°.

29. The apparatus of claim 19, wherein the top frustoconical surface and the bottom frustoconical surface comprise an upper base having a diameter of 1 mm to 10 mm.

30. The apparatus of claim 19, wherein each of the top conical or frustoconical surface and the bottom conical or frustoconical surface is independently composed of a material selected from the group consisting of stainless steel, poly(methyl acrylate), and glass.

31. The apparatus of claim 19, wherein the fluid inlet port is operably connected to a sample injection device.

32. The apparatus of claim 19, wherein:
(a) the top conical or frustoconical surface is actuated and the bottom conical or frustoconical surface is stationary; or
(b) the bottom conical or frustoconical surface is actuated and the top conical or frustoconical surface is stationary.

33. (canceled)

34. The apparatus of claim 19, wherein the bottom conical or frustoconical surface is configured to hold a fluid volume of at least between 10 μL and 100 μL.
